SEQUENCE LISTING (5 sequences)

SEQ ID NO: 1 - protein, 18 aa, Artificial
  peptide inhibitor
KKKLPATGDYMNMSPVGD

SEQ ID NO: 2 - protein, 7 aa, Artificial
  minimum consensus peptide sequence
LRRASLG

SEQ ID NO: 3 - protein, 16 aa, Artificial
  biopolymer-substrate mimetic
KKKLPATGDYMSPVGD

SEQ ID NO: 4 - protein, 30 aa, Artificial
  biopolymer-substrate mimetic
ARTKQTARKSTGGKAPRKQLATKAARKSAP

SEQ ID NO: 5 - protein, 30 aa, Artificial
  biopolymer-substrate mimetic
SGRGKGGKGLGKGGAKRHRKVLRDNIQGIT

(19) United States
(12) Patent Application Publication
(10) Pub. No.: US 2007/0264667 A1
(43) Pub. Date: Nov. 15, 2007

(54) Method and system for assaying transferase activity

(76) Inventors: Robert Gellibolian, Calabasas, CA (US); Riaz Rouhani, Concord, CA (US)

Correspondence Address: OLYMPIC PATENT WORKS PLLC, P.O. BOX 4277, SEATTLE, WA, 98104, US

(21) Appl. No.: 10/934,647

(22) Filed: Sep. 3, 2004

Related U.S. Application Data
(60) Provisional application No. 60/499,863, filed on Sep. 3, 2003.

(51) Int. Cl. G01N 33/53

(52) U.S. Cl. 435/7.1

(57) ABSTRACT

Embodiments of the present invention are directed to sensitive, specific, and commercially feasible assays for transferase activity. Various embodiments of the present invention include artificial, multifunctional substrates specific for particular transferases that are chemically altered by the transferases to produce easily detectable, modified, multifunctional substrates. In one class of embodiments, the artificial, multifunctional substrate comprises a small-molecule-substrate component, or small-molecule-substrate-analog component, linked by a linking component to a biopolymer-substrate-mimetic or biopolymer-substrate-analog component. At least two, generally well-separated reporter moieties are included in the artificial, multifunctional substrate. The transferase, for which the artificial, multifunctional substrate is designed to serve as an assay reagent, catalyzes a generally covalent modification of the artificial, multifunctional substrate to produce a modified, artificial, multifunctional substrate reaction product in which the two reporter moieties are closely positioned to one another. When closely positioned to one another, the reporter moieties are detectable by one of various instrumental techniques.

### CROSS-REFERENCE TO RELATED APPLICATION

This application claims the benefit of U.S. Provisional Application No. 60/499,863, filed Sep. 3, 2003.

### TECHNICAL FIELD

The present invention is related to assays for enzyme activity and, in particular, to assay methods, assay-reagent-development methods, and a class of assay reagents that allow for sensitive determination of transferase activity for a large class of transferases in any of a large variety of different types of sample solutions.

### BACKGROUND OF THE INVENTION

The term “transferase” refers to a large and very important class of enzymes that transfer chemical groups from one substrate to another. One very important subclass of the transferase class of enzymes includes transferases that transfer methyl, acetyl, glycosyl, phosphate, formyl, sulfur, ubiquinone, farensyl, sialyl, small-ubiquitin-like (“SUMO”), and other chemical groups from small-molecule substrates (“SMS”) to biopolymer substrates (“BS”) that include catalytic and regulatory proteins, ribonucleic-acid biopolymers, and deoxyribonucleic-acid biopolymers. The biopolymer-modifying reactions carried out by these transferases may be involved in the control and modulation of the cell-cycle, gene-expression, signal transduction, and a variety of other important biochemical and cellular mechanisms of, and responses exhibited by, living cells. For example, transferases which transfer a phosphate group from a nucleoside triphosphate to threonine, serine and tyrosine residues of catalytic and regulatory proteins, called “protein kinases,” are key components of many different cell-cycle regulating and intracellular and intercellular communications systems that, among other things, are involved in development, normal cell function, gene-expression regulation, and the onset and development of pathological conditions such as cancer. Over 500 distinct kinases, grouped into approximately 20 well-known classes, have so far been discovered. Protein kinases may be activated by various stimuli, including hormones, neurotransmitters, and growth factors, and may, in turn, activate myriad different types of proteins and other biopolymers, often in a series of cascading reactions that vastly amplify the original stimulus.

Because of their importance in contributing to a variety of pathophysiological states, including cancer, inflammatory conditions, autoimmune disorders, and cardiac diseases, and in regulating aspects of neoplasia, proliferation, invasion, angiogenesis and metastasis, protein kinases are attractive targets for research and drug development. For example, many pharmaceutical companies are currently seeking small-molecule-drug inhibitors of, and therapeutic agents directed to, particular protein kinases for study and treatment of various types of cancers and other diseases. Assays for particular protein kinases are needed for such research and drug-development efforts, but, so far, practical and sensitive assays specific for measuring the activity of specific protein kinases have been difficult to develop. Current technologies for assaying the activity of protein kinases include antibody-based assays and other techniques that require either expensive creation and isolation of antibody biopolymers specific to various phosphorylation products or that need reasonably large amounts of expensive substrate or substrate-analog reagents. Developing assays specific to a particular protein kinase is an especially difficult process, since many protein kinases catalyze reactions between chemically similar substrates, and since protein-kinase structures are highly conserved.

Other important types of transferases of particular, current research interest, and for which small-molecule drug inhibitors are being sought by pharmaceutical research efforts, include histone acetyl transferases taht acetylate lysine, methyl transferases that methylate lysine and arginine, and ubiquinyl transferases that ubiquinate lysine residues of histone proteins involved in gene-expression regulation. Covalent modification of histones provides an epigenetic marking system that represents a fundamental regulatory mechanism that appears to impact most chromatin-templated processes. This epigenetic marking system has far-reaching consequences for cell-fate decisions and for both normal and pathological cell development, and considerably extends the information-content potential of chromosomes above that of the nucleotide-sequence-based genetic code. Histone modifying transferases include histone acetyltransferases (“HATs”) and histone methyltransferases (“HMTs”). The latter class of enzymes includes protein arginine methyltransferases (“PRMTs”) and SET-domain HMTs, which are involved in methylating lysine residues in histones H3 and H4. The proteins within each of the HAT and PRMT families share a conserved catalytic core, but have little similarity outside the core catalytic domain, indicating both a commonality in the mechanism of chemical modification and a diversity in substrate specificities among the HAT and PRMT families. Many pharmaceutical and biotechnology companies are now actively pursuing programs for the identification and development of specific substrates and inhibitors to serve as biological tools for studying histone-modifying transferases as well as therapeutic agents targeting transferases responsible for various pathological conditions.

As cellular and the biochemical processes become further understood through additional research efforts, it is likely that many other types of transferases and transferase-mediated cellular and biochemical activities will be identified as important components of both normal and disease-related cellular functions, and sensitive, specific, and commercially feasible assays of these transferases will also be needed. Therefore, researchers, pharmaceutical companies, diagnosticians, and other professionals who work with transferase-mediated biochemical and cellular processes have recognized the need for sensitive, specific, and commercially feasible transferase assays.

### SUMMARY OF THE INVENTION

Embodiments of the present invention are directed to sensitive, specific, and commercially feasible assays for transferase activity. Various embodiments of the present invention include artificial, multifunctional substrates specific for particular transferases that are chemically altered by the transferases to produce modified, easily detectable, multifunctional substrates. In one class of embodiments, the artificial, multifunctional substrate comprises a small-molecule-substrate component, or small-molecule-substrate-analog component, linked by a linking component to a biopolymer-substrate-mimetic or biopolymer-substrate-analog component. At least two, generally well-separated reporter moieties are included in the artificial, multifunctional substrate. In one class of embodiments, the reporter moieties are chromophores. The transferase, for which the artificial, multifunctional substrate is designed to serve as an assay reagent, catalyzes a modification of the artificial, multifunctional substrate to produce a modified, artificial, multifunctional substrate reaction product in which the two reporter moieties are closely positioned to one another. When closely positioned to one another, the reporter moieties are detectable by one of various instrumental techniques. In the class of artificial substrates that include chromophore reporter molecules, the chromophores are detectable in the modified substrate by fluorescent resonance energy transfer (“FRET”) and/or by other related techniques. In another class of embodiments, a single reporter moiety that produces a detectable signal either before or after modification of the artifical, multifunctional substrate. In yet additional classes of embodiments, the artifical, multifunctional substrate may produce a detectable signal before or after modification, without the need for reporter moieties.

### BRIEF DESCRIPTION OF THE DRAWINGS

FIG. 1 abstractly illustrates the basic components of a general transferase-mediated biopolymer-modification reaction, the rate of which embodiments of the present invention are designed to measure.

FIG. 2 abstractly illustrates the transferase-mediated biopolymer-modification reaction, the rate of which is determined by embodiments of the present invention.

FIG. 3 illustrates a general approach to developing a specific artificial, multifunctional substrate and assay for a particular transferase that represents a class of embodiments of the present invention.

FIGS. 4A-C show an AMS developed for protein kinases assays.

FIGS. 5A and 5B show the chemical structures of Cy3B and Cy5.

FIG. 5C shows the complete biosubstrate-substrate-mimetic component for the AMS for protein kinase A.

FIG. 5D illustrates preparation of the Cy3B-labeled peptide.

FIG. 5E shows the linker component for the protein-kinase-A AMS.

FIG. 5F illustrates synthesis of (DMT)-^{Cy5}PM^{AcI}.

FIG. 5G illustrates synthetic steps in the synthesis of the Iodacetylhydrazine Linker.

FIG. 5H shows the linker component of the protein-kinase-A AMS covalently joined to the biopolymer-substrate-mimetic component of the protein-kinase-A AMS.

FIG. 5I shows synthetic steps in preparation of (DMT)PM^{Cy5}•Pep^{cy3B}.

FIG. 5J shows the small-molecule component of the protein-kinase-A AMS.

FIG. 5K shows synthetic steps in the synthesis of γ-(2-aminoethyloxy)-ATP.

FIG. 5L shows synthetic steps in the synthesis of ATP^{AcI}.

FIG. 5M shows the final fluorescent-biosensor AMS for protein kinase A.

FIG. 5N shows steps in the synthesis of ATP•PM^{Cy5}•Pep^{cy3B}. The (DMT)PM^{Cy5}•Pep^{cy3B} substrate is treated with a TFA solution (10-20%) in water.

FIG. 6A shows the biopolymer-substrate-mimetic component for the PRMT-1 AMS.

FIG. 6B shows the biopolymer-substrate-mimetic component for the PRMT-1 AMS covalently attached to the linker component for PRMT-1 AMS.

FIG. 6C shows the small-molecule component of the PRMT-1 AMS.

FIG. 6D shows the AMS for PRMT-1.

FIG. 7A shows the biopolymer-substrate component of the AMS for PCAF.

FIG. 7B shows the final PCAF AMS.

FIG. 8 shows atomic components of a generalized small-molecule component of various embodiments of an AMS.

### DETAILED DESCRIPTION OF THE INVENTION

Embodiments of the present invention are related to methods and artificial, multifunctional substrates used in the methods for assaying specific transferase activities. Specific embodiments of the present invention are directed to specific assays for the activities of specific protein kinases, histone acetyl transferases, and histone methyl transferases, three types of transferases to which significant research and drug-development efforts are currently being focused in academic-science and pharmaceutical communities. However, the general assay methods and assay-reagent-development methods of the present invention are applicable to designing and performing assays for any of a relatively broad and important class of transferases that modify biopolymer substrates, such as regulatory and synthetic proteins, ribonucleic-acid biopolymers, and deoxyribonucleic-acid biopolymers.

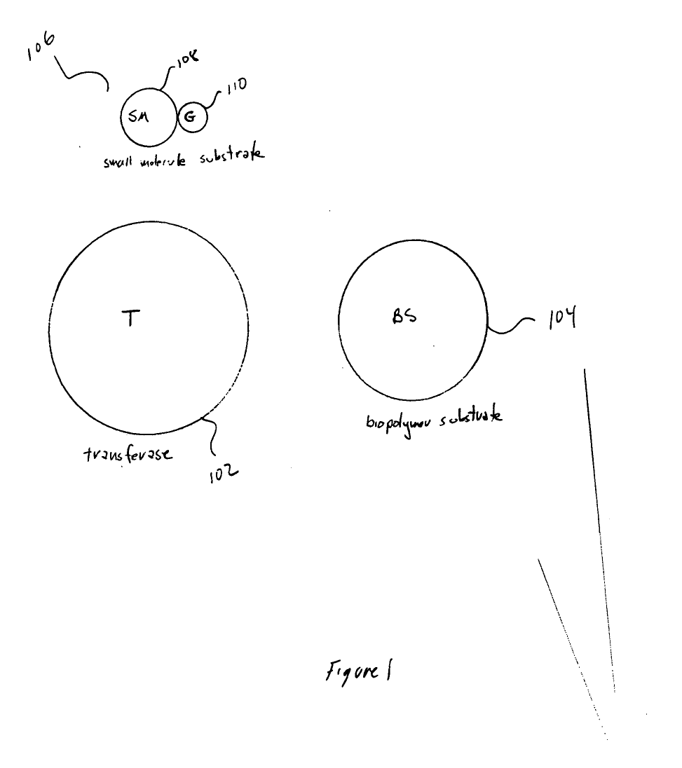

FIG. 1 abstractly illustrates the basic components of a general transferase-mediated biopolymer-modification reaction, the rate of which embodiments of the present invention are designed to measure. The reaction components include a transferase enzyme 102, a biopolymer substrate (“BS”) 104 modified by the transferase-mediated reaction, and a small-molecule substrate (“SMS”) 106 that includes a small molecule 108 covalently linked to a chemical group 110 via a generally relatively high-energy covalent bond. The high-energy nature of the bond between the small molecule 108 and the chemical group 110 ensures that breaking of the high-energy bond is exothermic, providing thermodynamically favored transfer of the chemical group to a molecule other than the small molecule to which it is attached in the SMS. In certain cases, the energy for transfer may be derived from an additional high-energy substrate, rather than from the functional-group-containing small-molecule substrate.

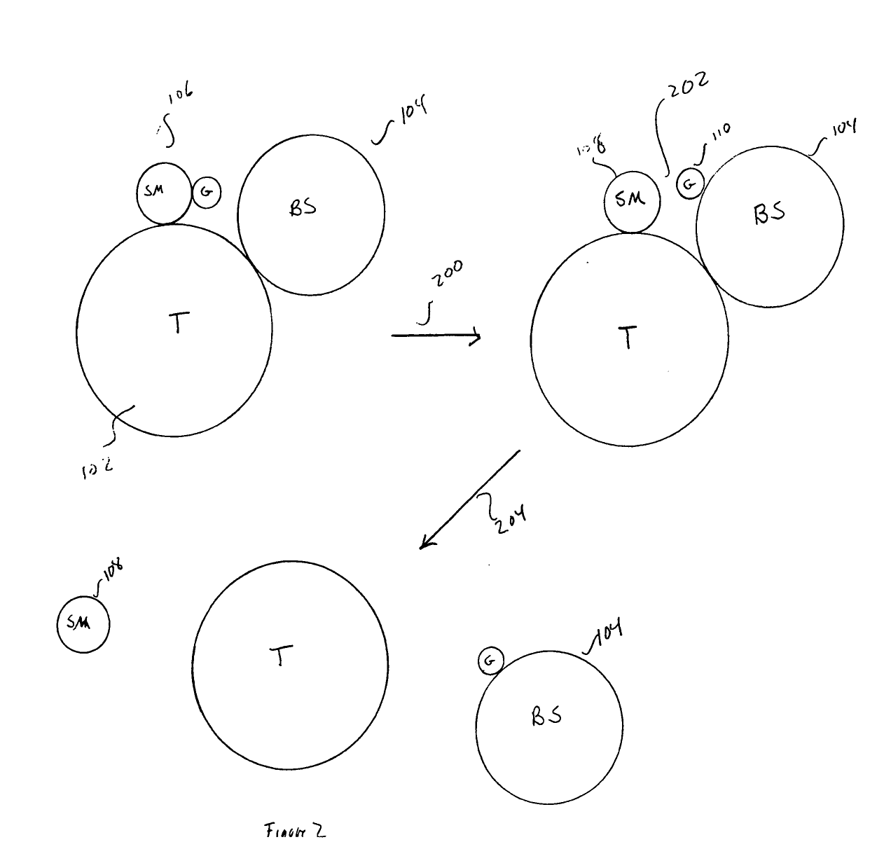

FIG. 2 abstractly illustrates the transferase-mediated biopolymer-modification reaction, the rate of which is determined by embodiments of the present invention. In FIG. 2, the SMS 106 and the BS 104 both bind to active sites of the transferase 102. In general, the active sites for the small-molecule substrate 106 and the biopolymer substrate 104 are in close proximity to one another within the transferase 102. In some transferases, the SMS may bind prior to the BS, in other transferases the BS may bind prior to the SMS, and in still other transferases, either order of SMS and BS binding may occur. Binding of one or both substrates may be passive, or may involve extraction of energy from an additional high-energy small molecule, such as a nucleoside triphosphate. Once the SMS and BS are bound to the transferase, the transferase catalyzes 200 cleavage of one or more covalent bonds joining the chemical group 110 to the small-molecule 108 component of the SMS 106, and transfers the chemical group to a target site of the biopolymer substrate 104. In FIG. 2, breaking of the one or more covalent bonds is shown by separation 202 of the small molecule 108 from the chemical group 110 and association of the chemical group 110 with the biopolymer substrate 104.

Various different types of reaction mechanisms may occur in the process of breaking the one or more bonds joining the chemical group 110 to the small molecule 108 and attaching the chemical group 110 to a target site within the biopolymer substrate 104. In certain cases, a single, concerted reaction mechanism may directly break and form bonds through a single transition-state intermediate. In other cases, the chemical group may be transferred first to a target site of the transferase, in a first concerted reaction, and then transferred from the target site of the transferase to the target site of the biopolymer substrate in a second concerted reaction. In certain cases, additional small-molecule cofactors may participate in one or more separate reactions that together transfer the chemical group from the SMS to the BS.

Finally, the small molecule 108 lacking the chemical group and the modified biopolymer substrate 104 containing the chemical group dissociate from the transferase to produce the three distinct products of the reaction in a second step 204 shown in FIG. 2. In certain cases, the reaction products disassociate in a particular order, while in other cases, the reaction products may disassociate in any order. Disassociation of the reaction products is generally a passive reaction, but, in certain cases, energy obtained from cleavage of a high-energy small molecule, such as a nucleoside triphosphate, may be used to actively dissociate one or more reaction products from the transferase.

As discussed above, there are many different types of transferases and transferase-mediated biopolymer-modification reactions. Table 3, below, lists a few of thousands of known transferases, along with the biopolymer substrate, small-molecule substrate, target site, and reaction products for the transferase.

| | | Small- | Trans- | |
|---|---|---|---|---|
| | Biopolymer | Molecule | ferred | Small |
| Transferase | Substrate | Substrate | Group | Molecule |
| protein | regulatory | ATP | phosphate | ADP |
| kinase | and catalytic | | | |
| | proteins | | | |
| histone | histone | SAM | methyl | SAH |
| methyl- | | | | |
| transferase | | | | |
| fatty-acid | fatty acid | SAM | methyl | SAH |
| O-methyl- | | | | |
| transferase | | | | |
| methionyl- | methionyl- | 10-formyl | formyl | tetrahy- |
| tRNA formyl | tRNA | tetrahy- | | drofolate |
| transferase | | drofolate | | |
| histone | histone | acetyl-CoA | acetyl | CoA |
| acetyl- | | | | |
| transferase | | | | |
| protein | protein | alkylamine | alkyl | NH₃ |
| glutamine | lysine | | | |
| α-glutamyl | residues | | | |
| transferase | | | | |
| DNA β- | DNA hydroxyl- | UDP- | β-glucose | UDP |
| glucosyl- | methylcytosine | glucose | | |
| transferase | moieties | | | |
| tRNA sulfur- | tRNA | L-cysteine | sulphhydryl | L- |
| transferase | | | | serine |

As can be seen in Table 3, above, different transferases can transfer a variety of different chemical groups from a variety of small-molecule substrates to a variety of different biopolymer substrates. Biopolymer substrates include proteins, ribonucleic acids, deoxyribonucleic acids, polysaccharides, glycoproteins, and lipids. Transferases may also transfer chemical groups from one small-molecule donor substrate to a different small-molecule substrate, such as the transferase thymidylate synthase, which transfers a methyl group from 5-10-methylenetetrahydrofolate to deoxyuridine monophosphate in order to produce deoxythymidine monophosphate. Small-molecule substrates include nucleotide triphosphates, S-adenosyl methionine, 10-formyl tetrahydrofolate, acetyl-CoA, UDP-glucose, cysteine, and many other small-molecule substrates. Groups transferred by transferases include phosphate, methyl, formyl, acetyl, glucose, sulfur, sulphate, alkyl, and many other types of chemical groups.

Many approaches to creating and practicing assays for transferase activity have been used. In one approach, antibodies that bind to modified biopolymer-substrates may be produced in order to facilitate determining the amount of modified biopolymer in a sample solution. It should be noted that a biopolymer substrate may undergo a relatively significant conformational change following transfer of a chemical group from a small molecule substrate to the biopolymer substrate, which, in turn, allows for creation of antibodies that recognize the modified-biopolymer-substrate conformation but not the unmodified-biopolymer-substrate conformation. Other techniques employ various characteristics of the small-molecule substrate, the small molecule following dissociation of the transferred group, modified small molecules or small-molecule substrates, or analogs of the small-molecule substrates, small molecules, or chemical groups in order to detect a decrease in the concentration of the small molecule substrate or an increase in reaction products. However, these latter types of assays may be insufficiently specific for particular transferases, and are often expensive, requiring significant amounts of assay reagents, including biopolymer substrates, in order to produce detectable levels of reaction products or a detectable decrease in reactants.

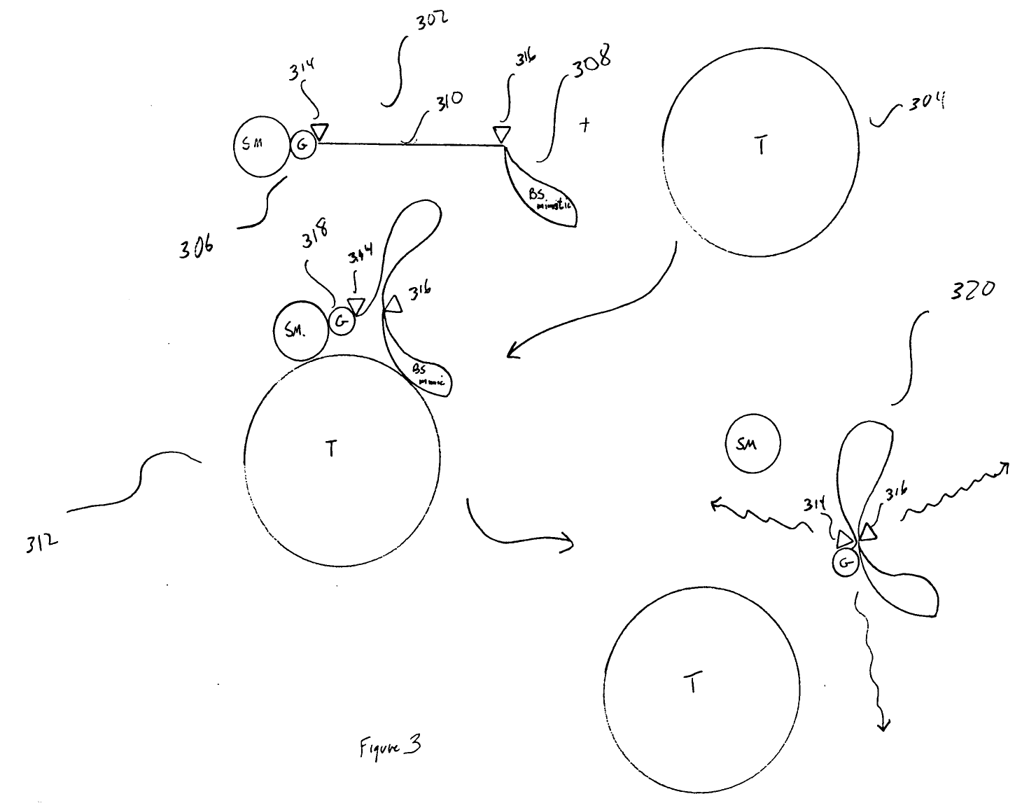

FIG. 3 illustrates one approach to developing a specific artificial, multifunctional substrate and assay for a particular transferase that represents a class of embodiments of the present invention. As shown in FIG. 3, the transferase assays that represent embodiments of the present invention employ an artificial, multifunctional substrate (“AMS”) 302 that specifically binds to, and is modified by, a target transferase 304. The AMS for a common, two-substrate transferase includes a small-molecule substrate component 306 joined to a biopolymer-substrate-mimetic component 308 by a linking component 310 and at least two reporter moieties 314 and 316. The small-molecule-substrate component 306 may be the normal SMS for the transferase, or may be an SMS analog that binds to the active site for the SMS in the transferase and from which the transferase can catalyze removal and transfer of a chemical group to produce a modified AMS that can be detected instrumentally. For example, when the transferase catalyzes transfer of a phosphate group from a neucleoside triphosphate substrate, this approach is not limited to the use of neucleoside triphosphates, but may include tetraphosphate derivatives of the normal nucleoside-triphosphate substrate. For example, a tetraphosphate derivative of ATP may extend the reactive phosphate group closer to the reactive serine/threonine/tyrosine group of the peptide that is modified by the transferase-mediated reaction and expose the reactive phosphate group to more of the solute environment of the kinase. In such cases, linkage chemistries will occur through the δ-phosphate of the nucleoside. Phosphate transfer in a tetra-phosphate derivative may occur from either the γ-phosphate or the δ-phosphate (transfer through the γ-phosphate leads to the pyrophosphate moiety being covalently attached to the reactive amino acid, whereas transfer from the δ-phosphate leads to a mono-phosphorylated species). To limit transfer to the δ-phosphate only, the tetraphosphate molecule can be derivatized to contain a non-reactive linkage (thiol-, amino-, CH₂, etc.), instead of a phosphodiester bond, between the β- and the γ-phosphates to limit transfer from the γ-phosphate.

The biopolymer-substrate-mimetic component 308 generally includes some portion of the biopolymer substrate, or analog to a portion of the biopolymer substrate, that binds to the active site for the biopolymer substrate in the transferase. Thus, the small-molecule-substrate component 306 of the AMS and the biopolymer-substrate-mimetic component 308 of the AMS both bind to the substrate binding sites of the transferase, as shown in FIG. 3 by the schematic representation of the bound AMS to the transferase 312.

The linking component 310 is a conformationally flexible covalent linker that correctly spaces the small-molecule-substrate component 306 of the AMS from the biopolymer-substrate-mimetic component 308 of the AMS for binding to the active sites of the transferase. The AMS includes a first reporter moiety 314 and a second reporter moiety 316. In the unbound, AMS 302, the two reporter moieties 314 and 316 are generally positioned relatively far apart, and the distance between the two reporter moieties constantly varies due to conformational flexibility of the linking component 310. In the unbound AMS, the two reporter moieties 314 and 316 are thus not positioned sufficiently closely and stably to permit a distance-sensitive interaction leading to a detectable signal. However, when bound to the transferase 312, due to the relatively stable conformation of the transferase and closely spaced binding sites for the SMS and BS-mimetic components of the AMS, the SMS component and BS-mimetic component of the AMS are relatively rigidly locked into closely separated active-site positions, placing reporter moieties 314 and 316 into relatively close proximity. The transferase then catalyzes transfer of the chemical group 318 or functional-group analog from the SMS component of the AMS to the BS-mimetic component of the AMS to produce a modified, AMS 320, releasing the modified artificial substrate and the small molecule or small-molecule-analog component as reaction products. The activity of transferase within the solution can be directly measured by measuring a signal generated from the reporter moieties held closely together within the modified AMS. In one class of embodiments, the reporter moieties are two different chromophores detected by fluorescent resonance energy transfer (“FRET”) in the modified, AMS reaction product.

There are a number of possible variations of the general reagent-assay-development method and assay method discussed above, with reference to FIG. 3. For example, while two different chromophores and the FRET technique can be used as the basis for detection of the modified AMS, and inference of transferase activity, other types of detectable modifications may be employed. For example, if two reporter groups, analogous to chromophores 314 and 316 of FIG. 3, form a spectroscopically detectable, low-energy intermediate when positionally fixed at relatively small distance from one another, the reporter molecules in the modified substrate can be spectroscopically detected. As another example, reporter groups with spin states detectable by NMR may show pronounced peak splitting when positionally fixed at a relative small distance from one another. As yet another example, cleavage of the AMS into an SMS leaving group and the BS-mimetic/linker components of the AMS may produce lower-mass products easily detectable by mass spectroscopy. One or more mass reporter groups may be used to enhance the difference between the AMS and AMS components following transferase-catalyzed cleavage of the SMS from the AMS. In certain embodiments, no, one, two, or more reporter groups may be used to provide a detectable signal in either the AMS or the cleavage and modification products of the AMS produced by the transferase-catalyzed reaction.

Although, in the described assay in FIG. 3, the modified AMS, lacking a portion of the small-molecule component, disassociates from the transferase, in alternative assays, a single modified AMS reaction product may be produced. In still additional embodiments, the artificial substrate may bind more or less irreversibly to the transferase, producing a detectable signal only in the bound state. In this assay, the absolute quantity of transferase in the sample solution may be determined. In many assays, additional small-molecule cofactors may be used, and potential inhibitors or drug-candidate molecules may be added to observe how transferase activity is affected by the inhibitors and/or drug candidates.

In general, the rate of increase of modified AMS is monitored instrumentally to determine a reaction rate vs. time profile from which a transferase activity can be determined for modification of the AMS, from which, in turn, a transferase activity for the normal substrates can be computed, using calibration standards. Assay solutions generally also include various buffers, anti-bacterial agents, salts, and other components that stabilize the sample solution at certain, well-known pH values, ionic strengths, and other such parameters and that prevent sample deterioration. To monitor protein modification, a wide variety of reaction conditions can be employed depending on the target enzyme in question. For example, the reaction for protein kinases generally takes place in incubation volumes of 50 μl or less and requires the presence of nM amounts of a kinase-specific fluorescent biosensor. For many of the mammalian kinases, the reaction is carried out at room or elevated temperatures, usually in the range of 20° to 40° C., but more conveniently at 25° C. For high-throughput applications, the reaction times are minimized to a range of 0.5 to 2 hours.

The AMS representing one embodiment of the assay-reagent invention of the present invention has several advantageous characteristics for transferase-activity measurement. First, the reaction kinetics for a multifunctional substrate may substantially improved with respect to normal binding of two or more discrete substrates needed to prime the transferase reaction. When, for example, two substrate components are covalently bound by a linker molecule, the effective concentration of the second substrate component following binding of the first substrate component is generally much higher, facilitating binding of the second substrate and greatly increasing the rate of substrate binding, often a limiting step in the transferase-mediated reaction. The equilibrium binding constants for the AMS may be significantly larger, due to the linkage between substrates. Thus, a far smaller amount of the AMS may be needed in order to produce a reliable, detectable signal than the concentrations needed for the normal substrates of the transferase-mediated reaction in currently used assay methods. As discussed above, a second advantage of the AMS is that modification by the transferase produces an easily instrumentally detectable reaction product, so that much lower amounts of reaction products need to be produced in order to produce a detectable signal. Finally, the small-molecule-substrate component and the biopolymer-substrate-mimetic component of the AMS may be chemically altered in order to produce more favorable binding kinetics and equilibrium binding constants when incorporated within an AMS. As a result of these advantageous characteristics, the AMS-based transferase assay of the present invention can be carried out with extremely small quantities of the AMS reagent, and the time course of the reaction may be significantly shortened, leading to both cost-efficient and time-efficient assays. Moreover, the AMS may be designed to specifically target a particular transferase, both by employing the normal SMS and a portion of the normal BS for the transferase in the AMS, and by tailoring the linking component to tailor the affinity of the artificial substrate to a particular transferase. Also, modified SMS components and BS-mimetic components may increase the specificity of the AMS for a particular substrate.

In certain alternate embodiments, the reporter moieties may be both attached to the linker component of the AMS, may be both attached to the BS-mimetic component of the AMS, or one reporter moiety may be attached to the chemical group transferred during the reaction or the linker component, and the other reporter moiety may be also attached to the linker or to the BS-mimetic component. It is important, in these embodiments, only that the reporter moieties be relatively widely separated in the AMS, but held relatively closely together in the modified AMS. By contrast, in still further embodiments, the reporter moieties may be closely spaced in the AMS, and relatively widely separated from one another in the modified AMS, so that an initially strong signal emitted by the AMS decreases, over time, as the AMS is modified by the transferase-mediated reaction.

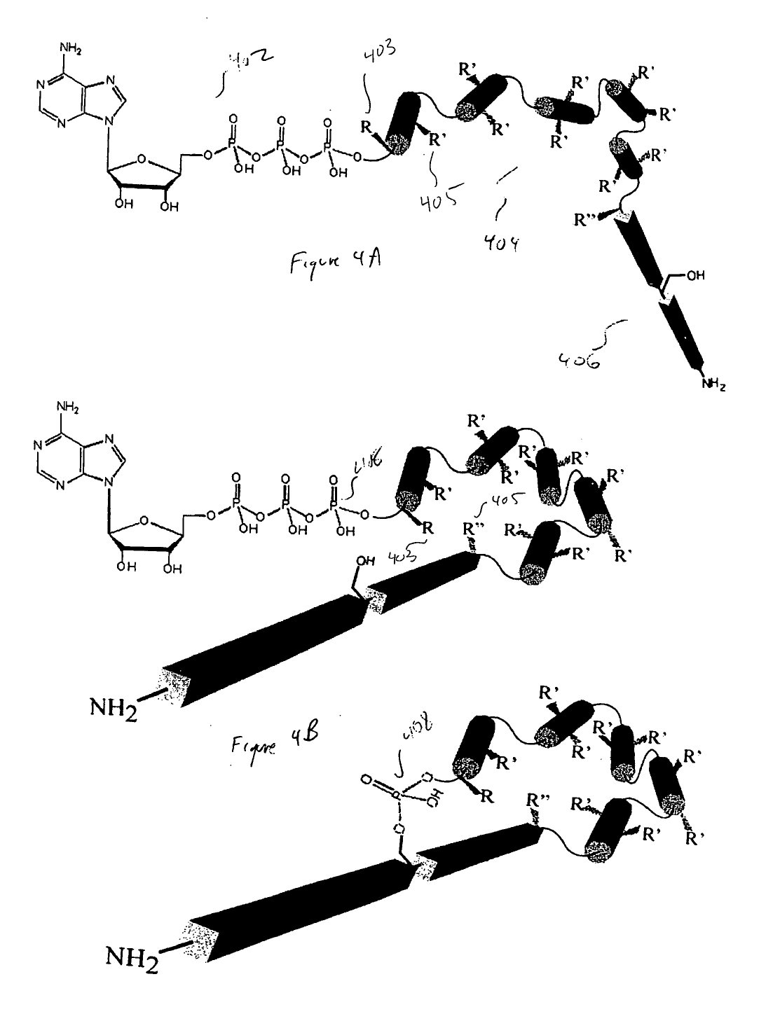

FIGS. 4A-C show an AMS developed for protein kinases assays. As can be seen in FIG. 4A, the AMS includes an ATP small-molecule-substrate component 402, a flexible linker component 404, and a biopolymer-substrate-mimetic component 405. Two chromophores R 403 and R″ 405 are attached to the ends of the linkers. In solution, prior to transferase modification, the linker component 404 is conformationally flexible, and the two chromophores R 403 and R″ 405 are therefore dynamically changing positions with respect to one another, but generally well separated from one another by the extended linker component. However, when bound to the active sites of the protein kinase, the positions of the small-molecule component and the biopolymer-substrate-mimetic component are fixed close to one another in the active sites, and the linker component is effectively constrained in a cyclic structure in which the two chromophores R 403 and R″ 405 are held in positions adjacent to one another, as shown in FIG. 4B. When the AMS is modified by transfer of the γ-phosphate 408 from the small-molecule component to the biopolymer-substrate component, as shown in FIG. 4C, the two chromophores are locked into adjacent positions by covalent cyclization of the linker component. When positioned close to one another, energy absorbed by one chromophore can be transferred to the other chromophore and then fluorescently emitted, leading to an easily detected fluorescent signal.

The approach represented by the many different embodiments of the present invention discussed above provides a high degree of mudlarity and provides for the state-space-search efficiencies of combinatoric, chemical-synthetic methods. Because the AMS is synthesized from 3 main components, any one or more of the components can be altered, in a systematic fashion, to generate different AMSs with different affinities for different transferases. Combinatoric synthesis of different AMSs provides a means for searching for as-yet unidentified transferases in complex biological sample solutions. An AMS may be rationally tailored to provide desirable properties with respect to on or a class of transferases by separately tailoring each of the three AMS components to a particular transferase.

### Detailed Embodiments

Protein Kinase Assays

To develop an assay to directly measure protein kinase activity, two fluorescent-biosensor AMSs for protein kinase A (“PKA”) and insulin receptor kinase (“IRK”) are synthesized and tested. The first AMS, _{Cy3B}^{Cy5}FS^{Kinase} (e.g., ATP•PM^{Cy5}•Pep^{cy3B}) links the γ-phosphate of aminoethyloxy-P^{γ}—O—P^{β}—O—P^{α}—O-5′-adenosine (e.g., ATP^{AcI}) to a peptidomimetic reporter group (i.e., PM) containing the two fluorescent probe pairs, Cy3B and Cy5, as well as the consensus peptide sequence of the protein kinase in question (e.g., ATP•PM^{Cy5}•Pep^{cy3B}). The second AMS, _{Cy3B}^{Cy5}FS2_{ATP+Pep}^{Kinase} comprises the Cy5-labeled ATP (ATP^{Cy5}), and the unlinked consensus peptide sequence of PKA or IRK with (or without) the attached Cy3B fluorophore near the phosphorylation site. The purpose of synthesizing two different classes of fluorescent-biosensor AMSs (i.e., linked versus unlinked) to measure protein kinase activity is to test the hypothesis that linking the two natural substrates, ATP and peptide, increases the binding efficiency to the kinase compared with either substrate alone. The choice of fluorophores is not limited to Cy3B and Cy5, but can involve any two combinations of fluorophore/quencher pairs with overlapping emission and excitation wavelengths.

This approach is not limited to ATP, but potentially can use any of the nucleotide tri-phosphates and tetra-phosphate derivatives. For example, tetra-phosphates tetra-phosphate derivatives extend and expose the reactive phosphate group to more of the solute environment and extend the reactive phosphate closer to the reactive serine/threonine/tyrosine group of the peptide being modified. In such cases, the linkage chemistries will occur through the δ-phosphate. Phosphate transfer in a tetra-phosphate derivative may occur from either the γ-phosphate or the δ-phosphate (transfer through the γ-phosphate leads to the pyrophosphate moiety being covalently attached to the reactive amino acid, whereas transfer from the δ-phosphate leads to a mono-phosphorylated species). To limit transfer to the δ-phosphate only, the tetra-phosphate molecule can be derivatized to contain a non-reactive linkage (thiol-, amino-, CH₂, etc.), instead of a phosphodiester bond, between the β- and the γ-phosphates to limit transfer from the γ-phosphate.

### 1. Synthesis of the Kinase Peptide Substrate, Pep^{Cy3B}

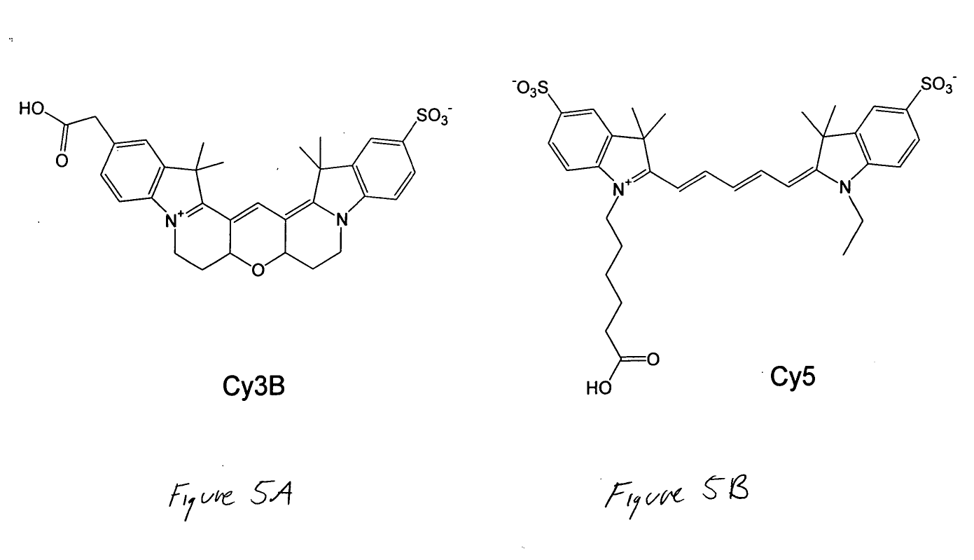

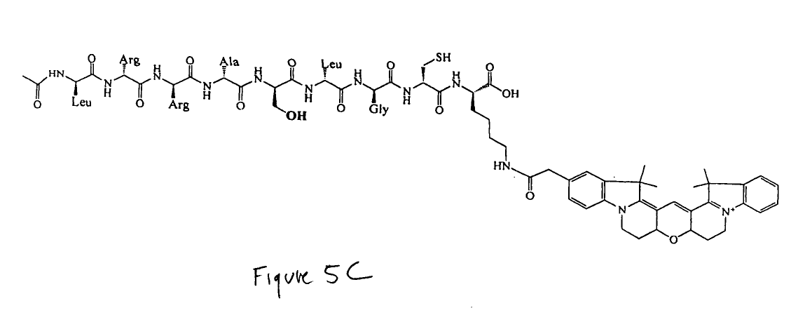

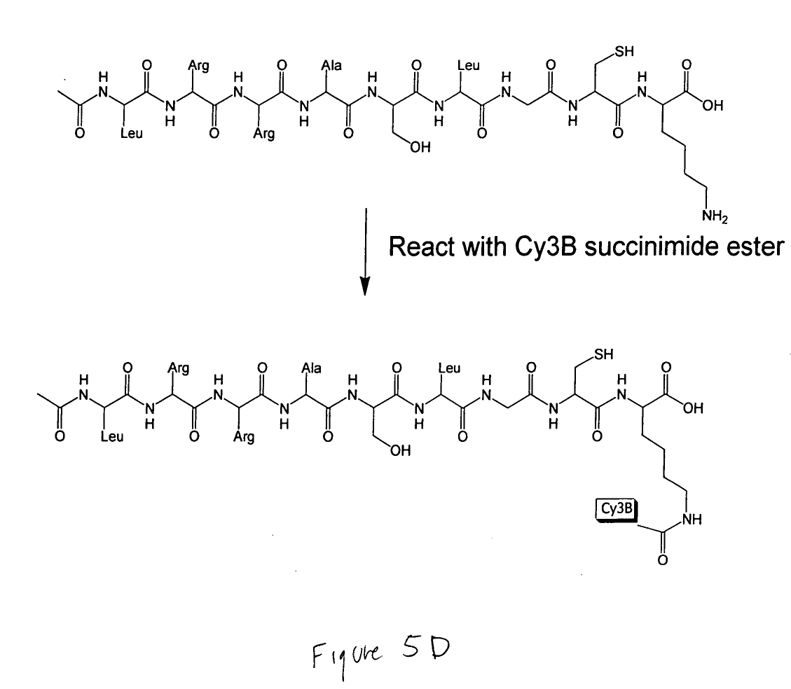

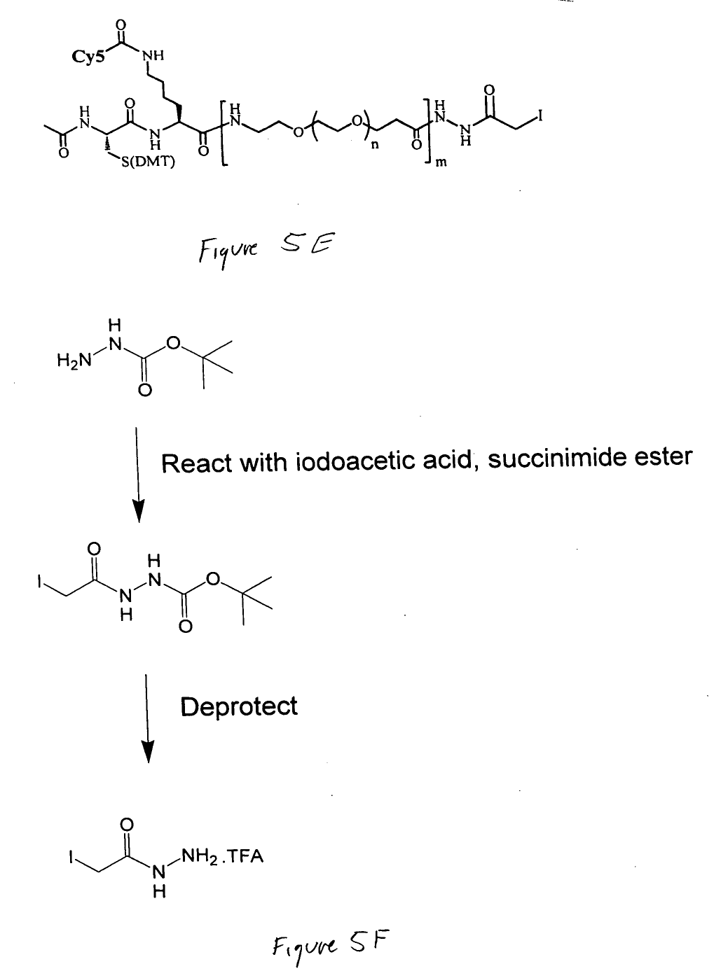

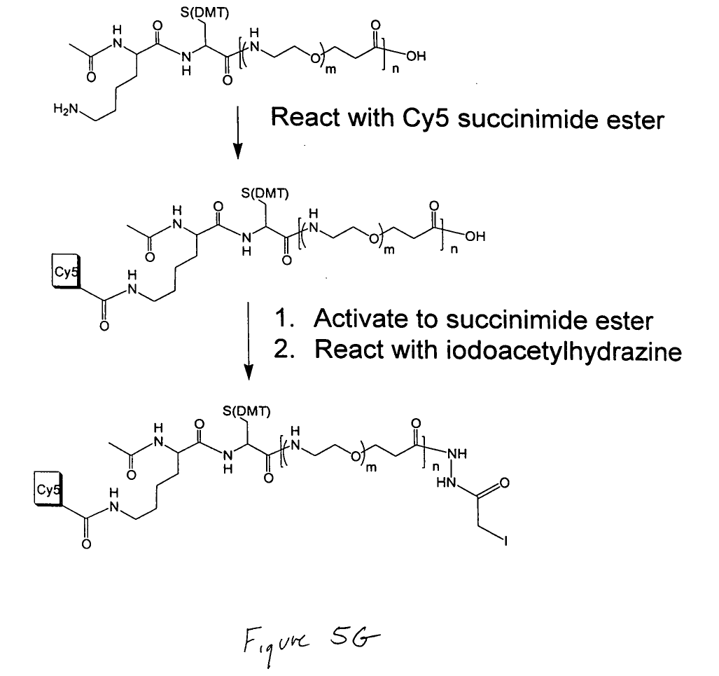

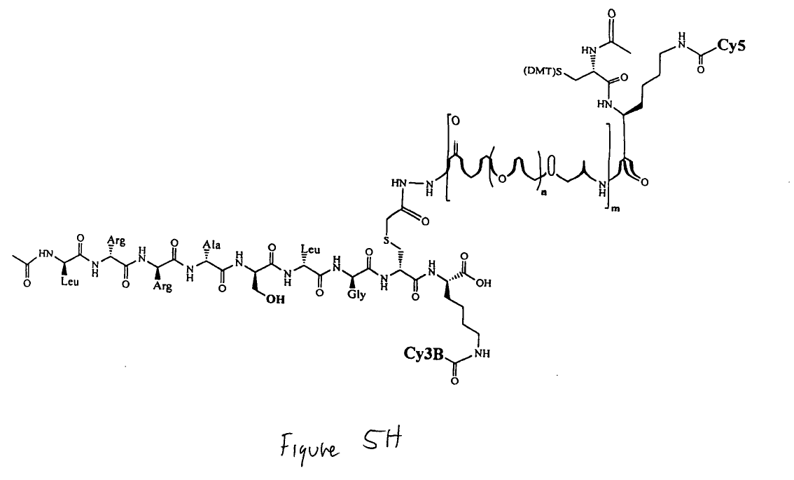

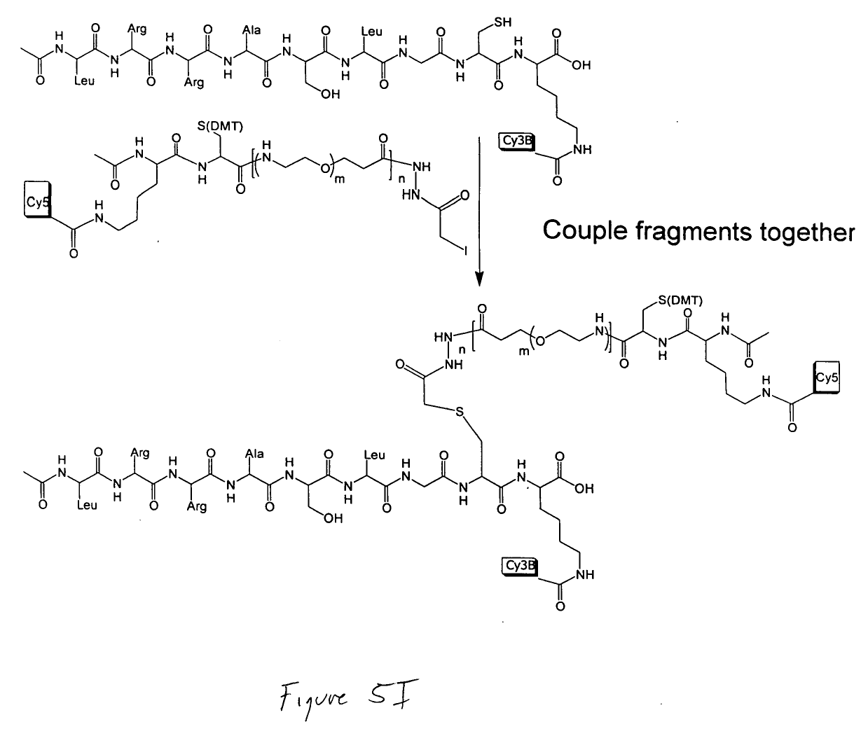

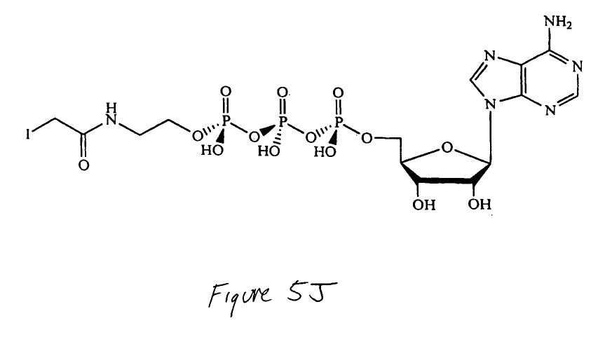

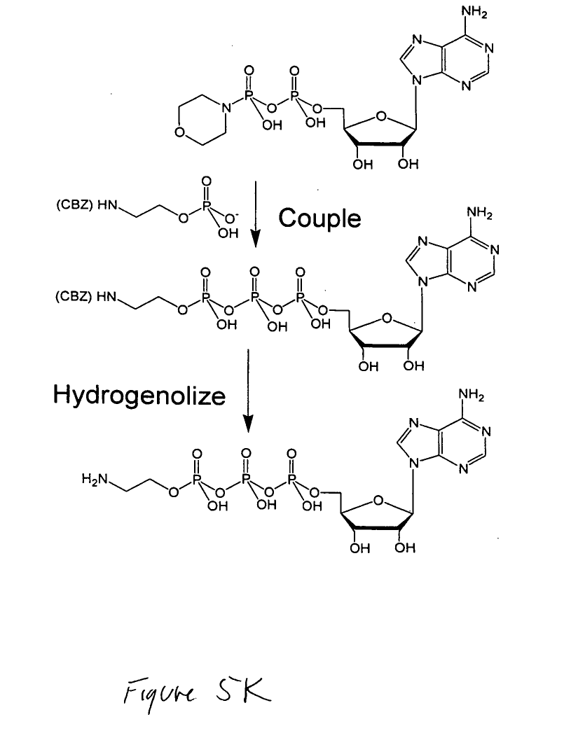

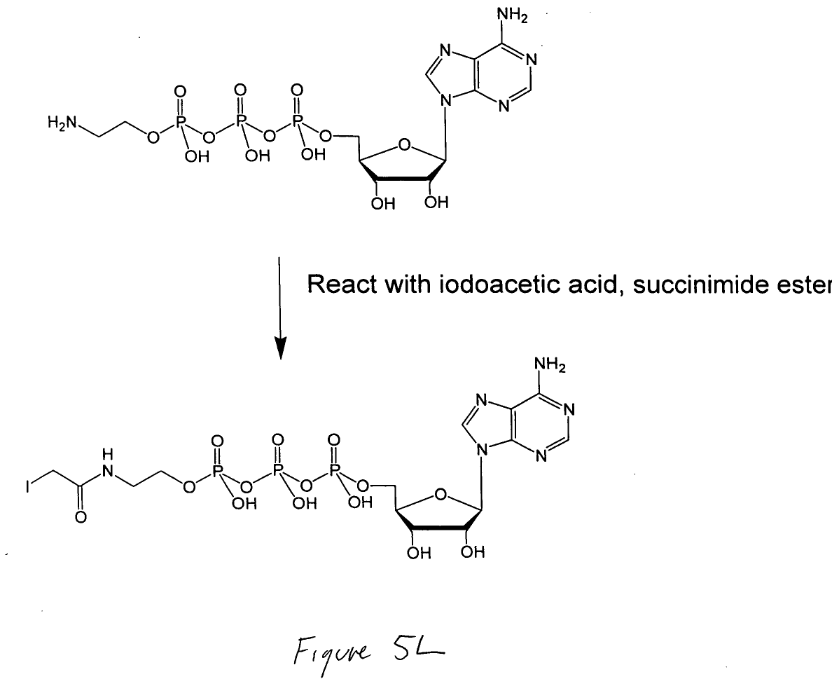

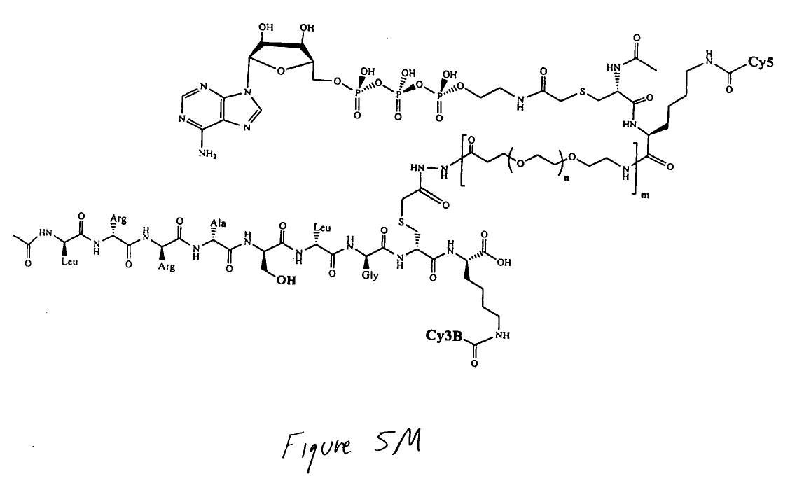

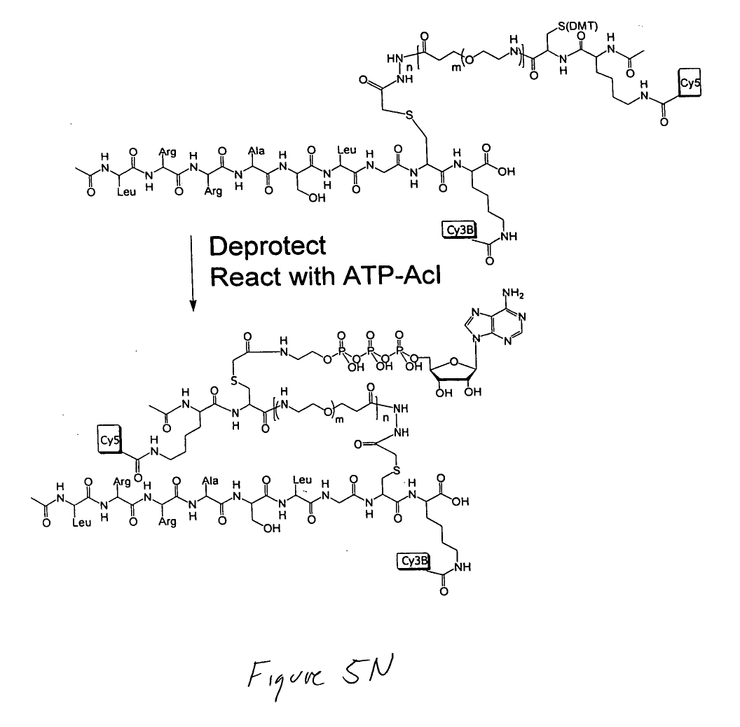

The peptide sequences for the biosubstrate-substrate-mimetic component for the AMS for PKA and IRK are prepared using standard Fmoc chemistry, with methods and conditions well known in the art. Appropriate preloaded resins and Fmoc amino acids are selected. FIGS. 5A and 5B show the chemical structures of Cy3B and Cy5. FIG. 5C shows the complete biosubstrate-substrate-mimetic component for the AMS for protein kinase A. The resin is either extended on an automated peptide synthesizer using the manufacturer's recommended protocols or manually using the following conditions:

### i. Peptide Synthesis

The resin is swollen with dichloromethane (DCM) and washed with dimethylformamide (DMF). The Fmoc protecting group is removed by treating the resin twice with 20% piperidine solution in the DMF for 10 minutes. The de-protected resin is coupled sequentially with Fmoc-Lys (“MTT”) and Fmoc-Cys (“TRT”). These amino acids are reserved for linking of the Reporter dye and the ATP reagents. The Fmoc protected amino acids are used to synthesize the kinase substrate sequence. Appropriate amounts of reagents such as HBTU or HATU are used to activate the amino acids in conjunction with HOBT and diisopropylethyl amine prior to coupling to the growing chain of the kinase sequence in dry DMF. Fmoc-Lys(Boc) is used for the rest of the lysine groups in the sequence to differentiate between the sequence-lysine and the linking-lysine. All the reactions are monitored with the Kaiser test for completion of the couplings. In case of an incomplete coupling, double coupling is performed. Final acetylation of the alpha amino group of the peptide chain terminus is performed if and when necessary.

### ii. Cleavage of the Peptide from the Resin

The peptide is consequently cleaved from the resin, using the standard cleavage cocktail such as TFA and scavengers such as phenol, thioanisol, ethanedithiol and water. Non-thiol scavengers such as triisopropylsilane (“TIPS”) can replace ethanedithiol. TFA is removed under vacuum. The de-protected peptide is precipitated by diethyl ether. The precipitated peptide is isolated and purified by HPLC, using a C18 reverse phase column as stationary phase and water: acetonitrile containing TFA gradient as mobile phase. The isolated purified peptide is analyzed by ESI-MS.

### iii. Coupling of Cy3B Dye to the Peptide

The purified peptide is reacted with the reactive ester of Cy3B, under an inert atmosphere and/or mild reducing condition. The reaction is monitored by HPLC. The final product is isolated by HPLC, using a C18 reverse phase column as stationary phase and water: acetonitrile mixture as the mobile phase. The isolated purified peptide is analyzed by ESI-MS. The proper fraction is lyophilized for long-term storage. FIG. 5D illustrates preparation of the Cy3B-labeled peptide.

### 2. Synthesis of the Reporter Arm, (DMT)-^{Cy5}PM^{AcI}

FIG. 5E shows the linker component for the protein-kinase-A AMS. The linker component is based on an extended polyethyleneglycol polymer. FIG. 5F illustrates synthesis of (DMT)-^{Cy5}PM^{AcI}. FIG. 5G illustrates synthetic steps in the synthesis of the Iodacetylhydrazine Linker.

### i. Preparation of the Fmoc-dPEGₙ-OH

ω-amino of the dPEGₙ™-tBu (Quanta Biodesign, Ltd., Powell, Ohio) is reacted with fluorenylmethylchloroformate in dichloromethane and triethylamine as base. The product Fmoc-dPEGₙ-O-tert-Bu is purified by flash column chromatography. The integrity of the structure is determined by NMR spectroscopy. Fmoc-dPEGₙ-O-tert-Bu is treated with neat TFA for 15 minutes. Trifluoroacetic acid is removed under high vacuum. The Fmoc-dPEGₙ-OH is used without further purification.

### ii. Preparation of Fmoc-dPEGₙ-Cl-Trityl

Chlorotrityl chloride resin is loaded with Fmoc-dPEGₙ-OH in DCM and diisopropylethylamine as base. The uptake is monitored by UV spectroscopy. Un-reacted resin is capped with acetate. Loading can be further determined by determination of the Fmoc released from the resin. The resin is washed with DCM and DMF and used without further characterization.

### iii. Preparation of the Peptoid Moiety of the Reporter Arm ((DMT)-^{H}₂^{N}PM^{CO}₂^{H})

Fmoc-dPEGₙ-Cl-trityl resin is further extended by repeated additions of the Fmoc-dPEGₙ-OH (1-5 cycles) by standard peptide synthesis as described above. Finally, Fmoc-Lys(MTT)-OH and Fmoc-Cys(TRT)-OH are added to the sequence. The alpha amino group of the sequence is capped off with acetic anhydride, using standard procedures known in the art. The sequence is finally cleaved off the resin, using standard cleavage protocols. The peptoid moiety is purified by HPLC and lyophilized. The isolated peptide is reacted with dimethoxytrityl chloride in DMF and triethylamine as base, purified by HPLC. The purified peptide is qualified by ESI MS analysis.

### iv. Coupling of Cy5 to the Lysine Residue of the Reporter Arm, ((DMT)-^{Cy5}PM^{CO}₂^{H})

To a solution of the purified (DMT)-^{H}₂^{N}PM^{CO}₂^{H} in DMF added a solution of the activated ester of Cy5 and triethylamine as base. Completion of the reaction is monitored by HPLC. The peptoid-Cy5 conjugate (i.e., (DMT)-^{Cy5}PM^{CO}₂^{H}) is purified by HPLC, using a C18 reverse phase column as stationary phase and water: acetonitrile mixture as the mobile phase and analyzed by ESI-MS.

### v. Preparation of the Functional Linker Arm, N-tert-butyloxycarbonyl-N-(iodoacetyl)-hydrazine, H₂N-AcI

Iodoacetic acid N-hydroxysuccinimide ester is reacted with tert-Butyl carbazate in DCM and triethylamine as base. The product is purified by flash column chromatography. Fractions are analyzed on a silica gel TLC plate. The product spot is correctly identified by treatment with hydrochloric acid followed by ninhydrine spray spot analysis. The correct fraction is isolated and analyzed by NMR spectroscopy. The BOC protecting group is removed prior to coupling to the peptoid moiety.

### vi. Preparation of (DMT)-^{Cy5}PM^{ACI} Peptoid Moiety

Cy5-peptoid (i.e., (DMT)-^{Cy5}PM^{CO}₂^{H}) is reacted with N-hydroxysuccinimide and N-(3-Dimethylaminopropyl)-N′-ethylcarbodiimide hydrochloride in DCM. The reaction progress is monitored by HPLC analysis. A sample of the Boc-HN—NH—COCH₂I is treated with TFA and the reaction allowed to proceed for 15 minutes. The TFA is removed under reduced pressure. The reagent 2-iodoacetyl hydrazine trifluoroacetate salt is added to the peptide solution in the presence of triethylamine. The reaction is monitored by HPLC. The final product is isolated by HPLC, using a C18 reverse phase column as stationary phase and water: acetonitrile mixture as the mobile phase. The isolated purified peptide is analyzed by ESI-MS. The proper fraction is lyophilized for long-term storage.

### 3. Preparation of the Kinase Peptidomimetic Substrate, ((DMT)PM^{Cy5}•Pep^{cy3B})-coupling of Cy5-peptoid with Cy3B-kinase Substrate

FIG. 5H shows the linker component of the protein-kinase-A AMS covalently joined to the biopolymer-substrate-mimetic component of the protein-kinase-A AMS. FIG. 51 shows synthetic steps in preparation of (DMT)PM^{Cy5}•Pep^{cy3B}. To a solution of Cy5-peptidoyl-iodoacetyl hydrazine in DMF and sodium phosphate buffer (pH 6.0-6.5) is added Cy3B-kinase substrate with free cysteine. The reaction is monitored by HPLC. The final product is isolated by HPLC, using a C18 reverse phase column as stationary phase and a water (containing triethylammonium acetate pH 5.5-6.0): acetonitrile mixture as the mobile phase. The isolated purified peptide is analyzed by ESI-MS. The proper fractions are lyophilized for coupling to the ATP reagent.

### 4. Preparation of the ATP Derivative, ATP^{AcI}

FIG. 5J shows the small-molecule component of the protein-kinase-A AMS. The small-molecule component is an ATP derivative. FIG. 5K shows synthetic steps in the synthesis of γ-(2-aminoethyloxy)-ATP. FIG. 5L shows synthetic steps in the synthesis of ATP^{AcI}.

### i. Preparation of the γ-(2-aminoethyloxy)-ATP

ADP-morpholidate is reacted with N-carbobenzyloxy-aminoethylphosphate in dry DMSO. The reaction progress is monitored by HPLC. The product is purified by ion exchange chromatography using triethylammonium hydrogen carbonate buffer gradient. The fraction containing the product are pooled and lyophilized. The product is analyzed by NMR and mass spectroscopy. The carbobenzyloxy group is removed by hydrogenolysis of the carbobenzyloxy-aminoethyloxy-ATP, using 5% Pd/C. Pd/C is filtered off and the solution is lyophilized, and stored at −80° C.

### ii. Preparation of the γ-(2-iodoacetyl aminoethyloxy)-ATP, ATP^{AcI}

A solution of γ-(2-aminoethyl)-ATP is reacted with iodoacetic acid N-hydroxysuccinimide ester in dimethylformamide with triethylamine as base. The reaction is monitored by HPLC. The final product is isolated by HPLC, using a C18 reverse phase column as stationary phase and water (containing triethylammonium acetate pH 5.5-6.0): acetonitrile mixture as the mobile phase. The isolated purified peptide is analyzed by ESI-MS. The proper fraction is lyophilized, and stored at −80° C.

### 5. Preparation of the Cy5-Labeled ATP Derivative, ATP^{Cy5}

### i. Preparation of the Cy5-Labeled Di-Peptide Conjugant, (DMT)-CK^{Cy5}

The di-peptide Cys-Lys is made on a resin by methods known in the art using Fmoc-Lys(MTT)-OH and Fmoc-Cys(TRT)-OH. The alpha amino group of the sequence is capped off with acetic anhydride, using standard procedures known in the art. The sequence is finally cleaved off the resin, using standard cleavage protocols. The di-peptide is purified by HPLC and lyophilized. The isolated peptide is reacted with dimethoxytrityl chloride in DMF and triethylamine as base, and purified by HPLC. The purified peptide is qualified by ESI MS analysis. To a solution of the purified di-peptide in DMF, we added a solution of the activated ester of Cy5 and triethylamine as base. Completion of the reaction is monitored by HPLC. The Cy5-di-peptide conjugate (i.e., (DMT)-CK^{Cy5}) is purified by HPLC, using a C18 reverse phase column as stationary phase and water: acetonitrile mixture as the mobile phase and analyzed by ESI-MS.

### ii. Preparation of the Cy5-Labeled ATP Derivative, ATP^{Cy5}

The (DMT)-CK^{Cy5} substrate is treated with a TFA solution (10-20%) in water. The deprotection reaction is monitored by HPLC. The HS-dipeptide-Cy5 is isolated by HPLC, using a C18 reverse phase column as stationary phase and water (containing triethylammonium acetate pH 5.5-6.0). The fraction containing the deprotected peptide is reacted with a solution of ATP^{AcI}. The reaction is monitored by HPLC. The final product is isolated by HPLC, using a C18 reverse phase column as stationary phase and water (containing triethylammonium acetate pH 5.5-6.0): acetonitrile mixture as the mobile phase. The isolated ATP^{Cy5} is analyzed by ESI-MS. The proper fraction is lyophilized, and stored at −80° C.

### 6. Synthesis of the Fluorescent-Biosensor AMSs for Protein Kinases, _{Cy3B}^{Cy5}FS^{Kinase} (ATP•PM^{Cy5}•pep^{cy3B})—coupling (DMT)PM^{Cy5}•Pep^{cy3B} to ATP^{AcI}

FIG. 5M shows the final fluorescent-biosensor AMS for protein kinase A. FIG. 5N shows steps in the synthesis of ATP.PM^{Cy5}•Pep^{cy3B}. The (DMT)PM^{Cy5}•Pep^{cy3B} substrate is treated with a TFA solution (10-20%) in water. The deprotection reaction is monitored by HPLC. After completion of the reaction the peptide is desalted on a G-25 sephadex column pre-equilibrated with pH 6.0-7.0 sodium phosphate buffer. The fraction containing the deprotected peptide is reacted with γ-(2-iodoacetylaminoethyloxy)-ATP. The reaction is monitored by HPLC. The final product is isolated by HPLC, using a C18 reverse phase column as stationary phase and water (containing triethylammonium acetate pH 5.5-6.0): acetonitrile mixture as the mobile phase. The isolated purified molecule is analyzed by ESI-MS. The proper fraction is lyophilized, and stored at −80° C.

### 7. Measuring Phosphoryl Transfer and Activity of PKA and IRK Protein Kinases

Two independent, non-overlapping assays can be employed to determine whether _{Cy3B}^{Cy5}FS^{PKA/IRK} can act as bonafide, potent substrate for measuring phosphoryl transfer using PKA or IRK kinases. One involves monitoring FRET between Cy3B and Cy5, as kinase activity transfers the γ-phosphate-linked Cy5 in close proximity to Cy3B in _{Cy3B}^{Cy5}FS^{PKA/IRK}. The second takes advantage of the fact that the chemical structure and composition of the substrate will change upon transfer of γ-phosphate to the consensus peptide sequence, facilitating the characterization and identification of the final product of phosphorylation using these synthetic substrates using mass spectrometry. Optimization of this assay involves determining the time course for _{Cy3B}^{Cy5}FS^{PKA/IRK} binding to PKA and IRK and the effects of varying the concentration of _{Cy3B}^{Cy5}FS^{PKA/IRK}, and the kinases.

To determine the viability of monitoring PKA and IRK protein kinase activity using the above-described AMSs, the binding of these reagents to the recombinant enzymes is assessed by steady-state kinetic analysis as exemplified by the single substrate of equation 1:

$$E + S ← \to ES {⟶}^{{k}_{cat}} EP$$

where E is the PKA or IRK;

S is the fluorescent biosensor reagent, _{Cy3B}^{Cy5}FS^{PKA/IRK};

ES is the enzyme bound reagent;

EP is the enzyme bound to the product; and

k_{cat} is the first-order rate constant for the conversion of ES to EP.

The experiments are performed under steady-state kinetics where the enzyme concentration (0.3-5 nM) is negligible compared to that of the substrate (30-500 nM).

This allows the calculation of the initial rate of product formation and/or destruction of substrate using the Michaelis-Menten equation:

$$v = \frac{[ {E}_{o} ] [ S ] {k}_{cat}}{{K}_{M} + [ S ]} = \frac{{V}_{max} [ S ]}{{K}_{M} + [ S ]}$$

or the linear form of equation 2, the Lineweaver-Burke plot:
1/v=1/Vₘₐₓ+K_{M}/Vₘₐₓ[S]
where v is the rate of product formation (FRET signal) and/or destruction of substrate (Cy5 and/or Cy3B signal);

[Eₒ] and [S] are the concentrations of enzyme and substrate, respectively;

Vₘₐₓ is the maximal velocity; and

K_{M} is the Michaelis constant, the concentration of substrate at which v=½Vₘₐₓ.

Experiments are performed under conditions of constant enzyme concentration, varying substrate concentration and constant time.

Non-specificity of binding is determined by measuring binding and turnover (as measured by FRET) of _{Cy3B}^{Cy5}FS^{PKA/IRK} to other protein kinases with different peptide sequence requirements. Optimization of conditions is carried out using the software JMP 5.0 (JMP, SAS Institute, Inc., Cary, N.Y.), and includes the determination of the kinetics of _{Cy3B}^{Cy5}FS^{PKA/IRK} binding to PKA and IRK under conditions of variable _{Cy3B}^{Cy5}FS^{PKA/IRK}, and recombinant enzyme. Secondary variables consist of buffer conditions (i.e., pH, salt, etc) and temperature.

Varying these conditions gives the highest signal to noise ratio for the assay and maximize reagent binding. In general, minimizing the volume of the reaction and amount of fluorescent biosensor favors optimal detection, but the actual concentrations and volumes depend on the binding affinity of the protein kinase in question. Phosphorylation of the peptide component of the fluorescent bioprobe is carried out using activated recombinant PKA (Upstate Group, Inc., Waltham, Mass.) and IRK (Affiniti research products, Ltd., Exeter, UK and A. G. Scientific, Inc., San Diego, Calif.). The reaction buffers are composed of 20 mM MgCl₂, 0.5 mM DTT, 0.05% BSA, 50 mM Tris-acetate, pH 7 for IRK and 20 mM MOPS, pH 7.2, 25 mM β-glycerol phosphate, 1 mM DTT, 5 mM EGTA, 1 mM Na orthovanadate for PKA. Protein kinase activity is assessed by FRET under conditions of varying pH, temperature, concentration of _{Cy3B}^{Cy5}FS^{PKA/IRK} and enzyme, using a fluorescent reader set up to record accumulation of signal at the emission wavelength of Cy5, λₑₘ^{Cy5}=667 nm, by excitation of the Cy3B at a wavelength of λₑₓ^{Cy3B}=566 nm.

### 8. Determination of Assay Specificity and Selectivity Using H89 and 1RS727 as Inhibitors of PKA and IRK

To determine the specificity of the assay, H89 (Upstate biotechnology, Waltham, Mass.) with an IC₅₀ of ˜50 mM, and 1RS727 (a peptide inhibitor with the sequence KKKLPATGDYMNMSPVGD) with a reported Kₘ of ˜24 μM (20), are used as potent competitive inhibitors of PKA and IRK, respectively. The experiments are performed with incubating each enzyme with varying concentrations of the appropriate inhibitor for 30 minutes, and performing the assay as previously described and the inhibition quantitated using the modified Lineweaver-Burke plot of equation 2 for competitive inhibition:

$$\frac{1}{v} = \frac{1}{{V}_{max}} + \frac{{K}_{M}}{{V}_{max} [ S ]} ( 1 + \frac{[ I ]}{{K}_{i}} )$$

where [I] is the concentration of the competitive inhibitor in the experiment; and Kᵢ is the inhibition constant.

To further investigate the specificity of the FRET assay in identifying selective inhibitors of PKA and IRK, the studies can be performed with the selective compounds of other protein kinases, with concentrations ranging from 1-1000 nM. The binding of _{Cy3B}^{Cy5}FS^{PKA/IRK} and to recombinant enzymes is tested using the highly selective cyclin-dependent protein kinase (CDPK) inhibitor Roscovitine (21). This compound is 100 fold more selective towards CDPK than any other kinase and exhibits and IC₅₀ towards PKC of over 100 μM. The potent (Kᵢ=11 nM) EGF receptor kinase inhibitor Lavendustin A (22) can also be tested in our assay, which like Roscovitine, has an IC₅₀ for PKC of >100 μM. The serine/threonine kinase inhibitors PD98059, SB202190 and U0126 (23-25), which selectively block MAP kinases, are also tested by the assay, as well as the cAMP dependent protein kinase inhibitor, PKI, for inhibition of FRET in our assay.

II. Histone Methyl-Transferase Reagents:

To develop an assay to directly measure HMT activity, a modular fluorescent-biosensor AMS for PRMT-1 methyl-transferase is synthesized and tested. The _{Cy3B}^{Cy5}FS^{PRMT-1} AMS links the thiol group of S-adenosyl-homocysteine to a peptidomimetic reporter moiety (e.g., (DMT)PM^{Cy5}.H4^{Cy3B}) containing the two fluorescent probe pairs, Cy3B and Cy5, as well as the N-terminal 30 amino acid residues of histone H4 (Note: H3 is not a substrate for this enzyme). _{Cy3B}^{Cy5}FS^{PRMT-1} measures the mono-methylation of Arg3 of histone H4 by PRMT-1 (26, 27). Like in the case of the protein kinases, the choice of fluorophores is not limited to Cy3B and Cy5, but can involve any two combinations of fluorophore/quencher pairs with overlapping emission and excitation wavelengths.

Moreover, this approach is not limited to the cofactor AdoMet, but potentially to any of the derivatives therein including, but not limited to those containing a different substituted group at the thiol position (i.e., amino-, CH₂, phosphorus, etc.).

### 1. Synthesis of the PRMT-1 Peptide Substrate. H₄^{Cy3B}

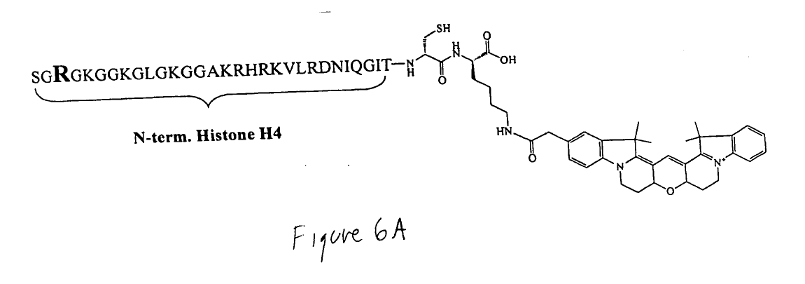

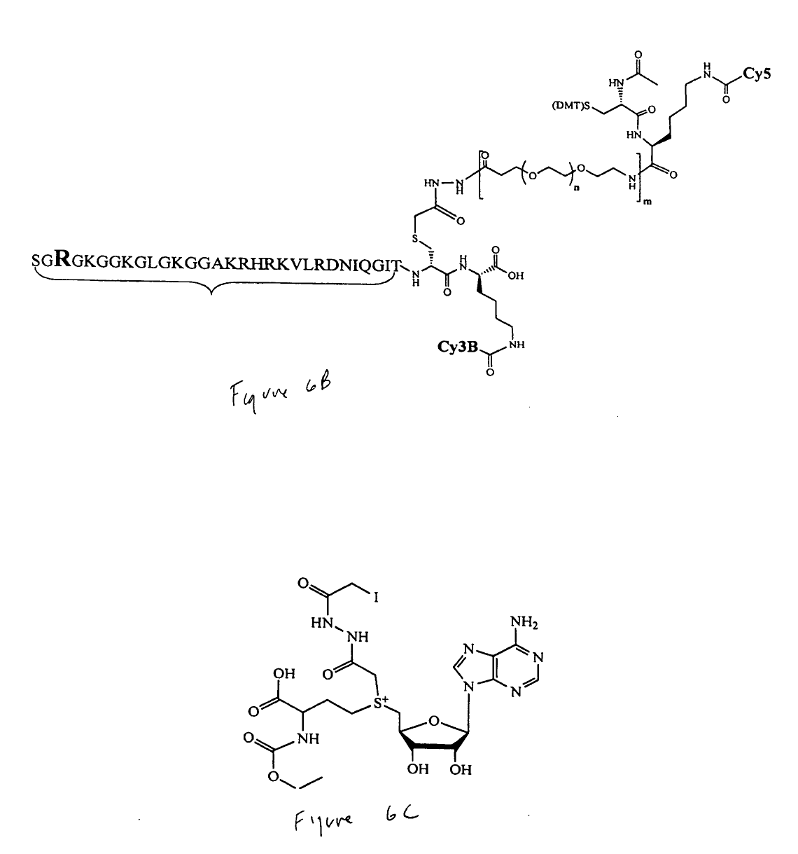

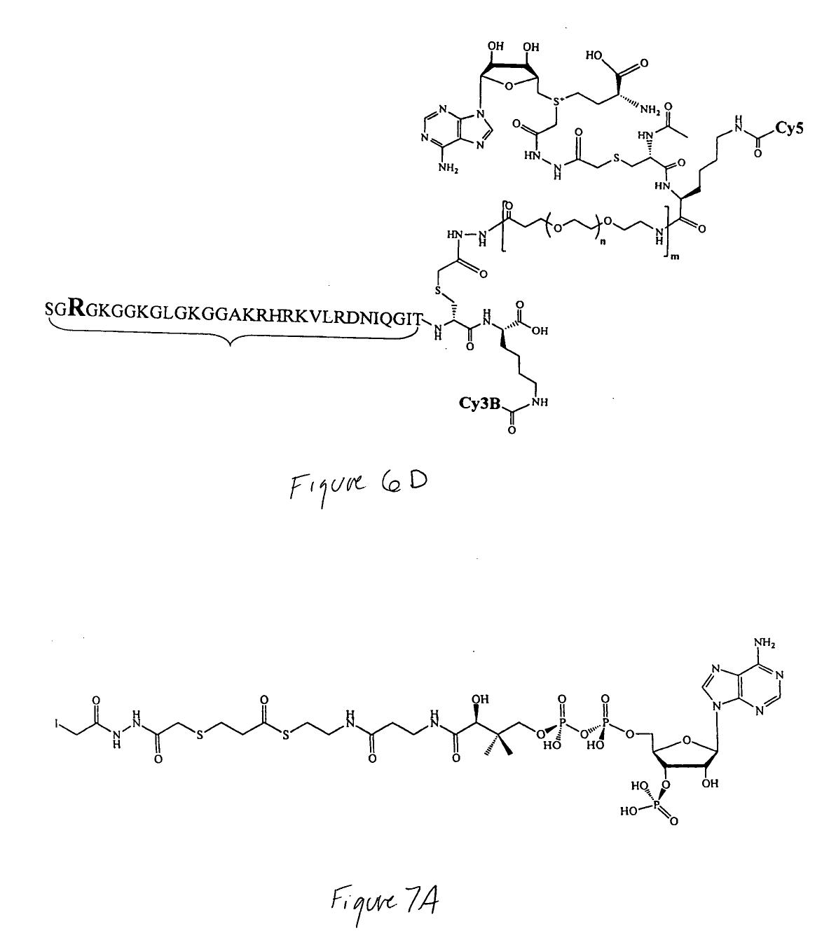

FIG. 6A shows the biopolymer-substrate-mimetic component for the PRMT-1 AMS. Similar to the preparation of PKA and IRK peptide substrates, the 30 amino acid residue peptide sequence of histone H4 tail, is prepared using standard Fmoc chemistry, with methods and conditions known in the art.

### 2. Preparation of the HMT Peptidomimetic Substrate, (DMT)PM^{Cy5}•H4^{Cy3B}—coupling of the Cy5-peptoid with H4^{Cy3B} peptide

FIG. 6B shows the biopolymer-substrate-mimetic component for the PRMT-1 AMS covalently attached to the linker component for PRMT-1 AMS. To a solution containing DMT-protected Cy5-peptidoyl-iodoacetyl hydrazine in DMF and sodium phosphate buffer (pH 6.0-6.5) is added Cy3B-labeled H4 peptide (H4^{Cy3B}) with free cysteine. The reaction is monitored by HPLC. The final product is isolated by HPLC, using a C18 reverse phase column as stationary phase and a water (containing triethylammonium acetate pH 5.5-6.0):acetonitrile mixture as the mobile phase. The isolated purified peptide is analyzed by ESI-MS. The proper fractions are lyophilized for coupling to the AdoMet reagent. (DMT)PM^{Cy5}•H4^{Cy3B} is treated briefly with TFA containing water to remove the DMT protecting group. TFA was removed by evaporation. The target SH—PM^{Cy5}.H4^{Cy3B} was purified by HPLC, using a C18 reverse phase column as the stationary phase and water: acetonitrile mixture as the mobile phase.

### 3. Preparation of the AdoMet Derivative. AM-10

FIG. 6C shows the small-molecule component of the PRMT-1 AMS. This small-molecule component is an adenosine derivative.

### i. Preparation of the N-2-trimethylsilylethyloxycarbonyl-hydrazine, (AM-1)

A cooled (−10° C.) solution of excess hydrazine hydrate (6-fold) and triethylamine (6 equivalents) in a mixture of acetonitrile: water (1:1) is added, dropwise, to a solution of 4-nitrophenyl-2-trimethylsilylethyl carbonate in acetonitrile. After complete addition of the acylating reagent, the mixture is stirred for 60 min. The solvent is removed under high vacuum. The residue is purified on a silica gel column. The final product N-(2-trimethylsilylethyl-oxycarbonyl) hydrazine (i.e., AM-1) is identified and analyzed by NMR and ESI-MS.

### ii. Preparation of the N-(2-iodoacetamido)-N′-(2-trimethyl-silylethyloxycarbonyl)-hydrazine (AM-2)

To a solution of the N-(2-trimethyl-silylethyloxycarbonyl) hydrazine (AM-1) (1 eq) in dimethylformamide is added 2-iodoacetic acid succinyl ester (1.1 eq). Triethylamine (1.1 eq) is added to the solution as base. The solution is stirred for 30 minutes. The solvent is removed under high vacuum. The product is purified on a silica gel column. The product N-(2-iodo-acetamido)-N′-(2-trimethyl-silylethyloxy-carbamoyl)-hydrazine (AM-2) is characterized by NMR and ESI-MS.

### iii. Preparation of N-tert-butyloxycarbonyl-S—(N′-acetamido-(N″-(2-trimethylsilylethyloxy-carbonyl))-hydrazinato)-homocysteine (AM-3)

A deoxygenated solution of the N-tert-butyloxycarbonyl-homocysteine (1 eq) in DMF is placed under an atmosphere of argon. To this are added sodium hydride (1 eq), and N-(2-iodo-acetamido)-N′-(2-trimethyl-silylethyloxy-carbonyl)-hydrazine (AM-2, 1.1 eq). The end of the reaction is determined by analysis of the reaction mixture by silica gel thin layer chromatography. The solvent is removed under high vacuum. The product N-tert-butyloxycarbonyl-S—(N′-acetamido-(N″-(2-trimethylsilyl-ethyloxycarbonyl))-hydrazinato)-homocysteine (AM-3) is purified on a silica gel column and analyzed by NMR spectroscopy.

### iv. Preparation of 5′-dimethoxytrityl-adenosine (AM-4)

To a solution of anhydrous adenosine (1 eq) in dry dimethylformamide was added dimethoxy-tritylchloride (1.1 eq) and triethylamine (1.1 eq). The solution is stirred for 60 minutes. DMF was removed under high vacuum at room temperature. The final product 5′-dimethoxytrityl-adenosine (AM4) is purified on a silica column. The purified product is identified by NMR spectroscopy.

### v. Preparation of 5′-dimethoxytrityl-N-trimethylsilylethyloxycarbonyl-2′,3′-(bis(2-trimethylsilylethyloxy-carbonyl))-adenosine (AM-5)

To a solution of the 5′-dimethoxytrityl-adenosine (AM4, 1 eq) are added sodium hydride (3.2 eq) and dry DMF. The mixture is stirred for 15 minutes. To this was added 4-nitrophenyl-2-trimethylsilylethyl carbonate (3.2 eq). The end of the reaction is determined by analysis of the reaction mixture by thin layer chromatography. The solvent is removed under high vacuum. The product 5′-dimethoxytrityl-N-trimethylethyl-oxycarbamoyl-2′,3′-(bis(2-trimethylethyloxy-carbonyl))adenosine (AM-5) is purified on a column of silica gel, and is identified by NMR spectroscopy.

### vi. Preparation of N-trimethylsilylethyl-oxycarbonyl-2′,3′-(bis(2-trimethyl-silylethyloxycarbonyl))adenosine (AM-6)

To a solution of 5′-dimethoxytrityl-N-(2-trimethylsilylethyloxycarbonyl-2′,3′-(bis(2-trimethyl-ethyloxycarbonyl))adenosine (AM-5) in acetonitrile was added equal volume of a 10% solution of acetic acid in water. The reaction is monitored by silica gel TLC analysis. The solvent is removed under reduced pressure at room temperature. The product N-trimethyl-silylethyloxy-carbonyl-2′,3′-(bis(2-trimethylsilyl-ethyloxycarbonyl))adenosine (AM-6) is purified on a column of silica gel, and is identified by NMR spectroscopy.

### vii. Preparation of 5′-methanesulfonyl-N-trimethylsilylethyloxycarbonyl-2′,3′-(bis(2-trimethylsilylethyloxy-carbonyl))adenosine (AM-7)

To a solution of N-(2-trimethylsilyl-ethyloxycarbonyl-2′,3′-(bis(2-trimethylethyloxy-carbonyl))adenosine (AM-6) in dry dichloro-methane is added methanesulfonylchloride (1.1 eq) and triethylamine (1.1 eq). The reaction is monitored by TLC analysis. The solvent is removed under reduced pressure at room temperature. The product 5′-methanesulfonyl-N-trimethylsilylethyl-oxycarbonyl-2′,3′-(bis(2-tri-methylsilylethyloxycarbonyl))adenosine (AM-7) is purified on a column of silica gel, and identified by NMR spectroscopy.

### viii. Preparation of 5′,S—(N-(tert-butyloxycarbonyl-S—(N′-acetamido-(N″-(2-trimethylsilylethyl-oxycarbonyl))-hydrazinato)-homocysteinyl)-N-trimethylsilylethyl-oxycarbonyl-2′,3′-(bis(2-trimethyl-silylethyloxycarbonyl))adenosine (AM-8)

To a solution of 5′-methanesulfonyl-N-trimethylsilylethyloxycarbonyl-2′,3′-(bis(2-tri-methylsilylethyloxycarbonyl))adenosine (AM-7, 1 eq) in dry DMF is added N-tert-butyloxycarbonyl-S—(N′-acetamido-(N″-(2-tri-methylsilyl-ethyloxycarbonyl))-hydrazinato)-homocysteine (AM-3, 1.1 eq). The reaction is heated at 80° C. The completion of the reaction is monitored by silica gel TLC analysis. After completion the solvent is removed under high vacuum. The product is purified on a column of silica gel. The product 5′,S—(N-(tert-butyloxycarbonyl-S—(N′-acetamido-(N″-(2-trimethylsilyl-ethyloxy-carbonyl))-hydrazinato) homocysteinyl)-N-trimethylsilyl-ethyloxy-carbonyl-2′,3′-(bis(2-trimethylsilylethyloxy-carbonyl))adenosine (AM-8) is purified on a column of silica gel, and is identified by NMR spectroscopy.

### ix. Preparation of 5′,S—(N-(tert-butyloxy-carbonyl-S—(N′-acetamido)-hydrazinato)-homocysteinyl)adenosine (AM-9)

To a solution of 5′,S—(N-(tert-butyloxycarbonyl-S—(N′-acetamido-(N″-(2-trimethylsilylethyloxy-carbonyl))-hydrazinato)-homocysteinyl)-N-tri-methylsilylethyl-oxycarbonyl-2′,3′-(bis(2-tri-methylsilylethyloxycarbonyl))adenosine (AM-8) in acetonitrile is added an aqueous solution of tetrabutyl-ammonium fluoride. The deprotection reaction is monitored by HPLC on a reversed phase C18 column. After the completion of the reaction the mixture is purified by HPLC. The product, 5′,S—(N-(tert-butyloxycarbonyl-S—(N′-acetamido)-hydrazinato)-homocysteinyl)adenosine (AM-9) is lyophilized and characterized by ESI-MS.

### x. Preparation of 5,S—(N-(tert-butyloxy-carbonyl)-(S—(N′-acetamido)-N″-(2-iodoacetamido)-hydrazinato)-homocysteinyl)adenosine (AM-10)

To a solution of 5′,S—(N-(tert-butyloxy-carbonyl-S—(N′-acetamido)-hydrazinato)-homocysteinyl)adenosine (AM-9, 1 eq) in dry DMF was added 2-iodoacetic acid succinyl ester (1.1 eq) and triethylamine (1.1 eq). The reaction is monitored by HPLC on a reversed phase C18 column. After the completion of the reaction the mixture is purified by HPLC on a C18 reverse phase column with triethylammonium acetate (100 mM, pH 5.5). The product, 5′,S—(N-(tert-butyloxy-carbonyl)-(S—(N′-acetamido)-N″-(2-iodoacetamido)-hydrazinato)-homocysteinyl)adenosine (AM-10) is lyophilized and characterized by NMR and ESI-MS.

### 4. Synthesis of the Fluorescent Biosensor for PRMT-1, _{Cy3B}^{Cy5}FS^{PRMT-1}

FIG. 6D shows the AMS for PRMT-1. The (DMT)PM^{Cy5}.H₄^{Cy3B} substrate is treated with a TFA solution (5-10%) in water. The deprotection reaction is monitored by HPLC. After completion of the reaction the peptide is desalted on a G-25 sephadex column pre-equilibrated with pH 6.0-7.0 sodium phosphate buffer. The fraction containing the deprotected peptide is reacted with 5′,S—(N-(tert-butyloxy-carbonyl)-(S—(N′-acetamido)-N″-(2-iodo-acetamido)-hydrazinato)-homocysteinyl)adenosine (e.g., AM-10). The reaction is monitored by HPLC. The final product is isolated by HPLC, using a C18 reverse phase column as stationary phase and water (containing triethyl-ammonium acetate pH 5.5-6.0): acetonitrile mixture as the mobile phase. The isolated purified peptide is analyzed by ESI-MS. The proper fraction is lyophilized, and stored at −80° C.

### 5. Measuring Methyl Transfer and Activity of PRMT-1 Histone Methyl-Transferase

Like protein kinases, two independent, non-overlapping assays is employed to determine whether _{Cy3B}^{Cy5}FS^{PRMT-1} can act as bonafide, potent substrate for measuring methyl transfer using PRMT-1 histone methyl-transferase. One involves monitoring FRET between Cy3B and Cy5, as methylation of the peptide substrate transfers the derivatized methyl-linked Cy5 in close proximity to Cy3B in _{Cy3B}^{Cy5}FS^{PRMT-1}. The second takes advantage of the fact that the chemical structure and composition of the substrate changes upon transfer of γ-phosphate to the consensus peptide sequence, facilitating the characterization and identification of the final product of phosphorylation using these synthetic substrates using mass spectrometry. Optimization of this assay involves determining the time course for _{Cy3B}^{Cy5}FS^{PRMT-1} binding to PRMT-1 and the effects of varying the concentration of _{Cy3B}^{Cy5}FS^{PRMT-1}, and the kinases. As before, the Michaelis-Menten and Lineweaver-Burke equations are used to analyze the experimental results.

Methylation of the peptide component of the fluorescent bioprobe is carried out using activated recombinant PRMT-1 (Cat# 14-474, Upstate Group, Inc., Waltham, Mass.). The recommended reaction buffers (50 mM Tris-HCl (pH 9.0), 0.5 mM DTT, 1 mM PMSF) is used for PRMT-1. Protein kinase activity is assessed by FRET under conditions of varying pH, temperature, concentration of _{Cy3B}^{Cy5}FS^{PRMT-1} and enzyme, using a fluorescent reader set up to record accumulation of signal at the emission wavelength of Cy5, λₑₘ^{Cy5}=667 nm, by excitation of the Cy3B at a wavelength of λₑₓ^{Cy3B}=566 nm.

III. Histone Acetyl-Transferase Reagents:

To develop an assay to directly measure histone acetylation, a fluorescent sensor is synthesized for monitoring the activity of PCAF. This substrate (^{PCAF}FS^{H3/H4}) is designed to measure the specific acetylation of Lys14 of histone H3 and/or Lys8 of histone H4 by PCAF.

### 1. Synthesis of the PCAF Peptide Substrates, H4^{Cy3B} and H3^{Cy3B}

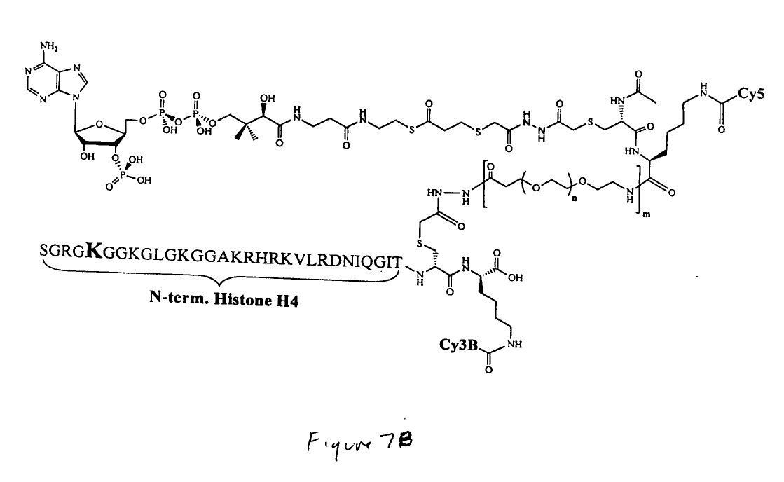

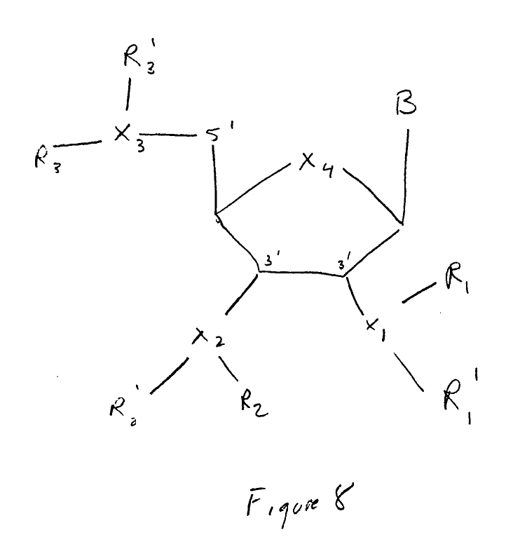

FIG. 7A shows the biopolymer-substrate component of the AMS for PCAF. Both H3 and H4 N-terminal tails are substrates for PCAF histone acetyltransferase. The targeted residues are Lys 18 and Lys4 of H3 and H4, respectively. The 30 amino acid residue N-terminal H3 and H4 sequences are prepared using Fmoc chemistry under the conditions known in the art as described previously in section I.

### 2. Preparation of the Acetyl-Transferase Peptidomimetic Substrate. (PM^{Cy5}•H4^{cy3B})—coupling of Cy5-peptoid with H4^{Cy3B} substrate:

### i. Preparation of (DMT)PM^{Cy5}•H4^{cy3B}

To a solution of (DMT)-^{Cy5}PM^{AcI}, we added a solution of H4^{Cy3B} substrate dissolved in DMF with free cysteine in sodium phosphate buffer (pH 6.0-6.5). The reaction is monitored by HPLC. The final product is isolated by HPLC, using a C18 reverse phase column as stationary phase and water (containing triethylammonium acetate pH 5.5-6.0): acetonitrile as mobile phase. The isolated purified peptide ((DMT)PM^{Cy5}•H4^{cy3B}) is analyzed by ESI-MS. The proper fractions are lyophilized for coupling to the Acyl coenzyme A reagent.

### ii. Preparation of PM^{Cy5}•H4^{cy3B}

DMT protected dye (DMT)PM^{Cy5}•H4^{cy3B} is treated briefly with trifluoroacetic acid containing water and dithiothreitol (DTT) as scavenger. Trifluoroacetic acid was removed by evaporation. The target PM^{Cy5}•H4^{cy3B} peptide is purified by HPLC, using a C18 reverse phase column as stationary phase and water: acetonitrile as mobile phase. The correct fraction is identified by mass analysis and lyophilized for future use.

### 3. Preparation of the Acetyl-CoA Derivative

### i. Preparation of the 3-(S-diphenyl-4-pyridylmethyl)mercaptopropionic acid (AC-1)

To a solution of mercaptopropionic acid in dichloromethane is added diphenyl-4-pyridylmethanol. Catalytic amounts dowex-X8 (H⁺) is added to the solution. The completion of the reaction is monitored by thin-layer chromato-graphy. The product 3-(S-diphenyl-4-pyridylmethyl)mercaptopropionic acid (AC-1) is purified by silica-gel chromatography, and identified by NMR and mass spectral analysis.

### ii. Preparation of the pentafluorophenyl 3-(S-diphenyl-4-pyridylmethyl)mercaptopropionic Acid (AC-2)

To a solution of 3-(S-diphenyl4-pyridylmethyl)mercaptopropionic acid (AC-1) in anhydrous dichloromethane is added 2,3,4,5,6-pentaflurophenol (1.1 eq) and dicyclohexylcarbodiimide (DCC, 1.1 eq). The completion of the reaction is monitored by thin-layer chromatography. The product pentafluorophenyl 3-(S-diphenyl-4-pyridylmethyl)mercaptopropionate (AC-2) is purified by silica-gel chromatography, and identified by NMR and mass spectral analysis.

### iii. Preparation of the 3-(S-diphenyl-4-pyridylmethyl)mercapto-propionyl Coenzyme A (AC-3)

To a solution of to a solution of coenzyme A (triethylammonium salt) in anhydrous dimethylsulfoxide (DMSO) was added pentafluorophenyl 3-(S-diphenyl-4-pyridylmethyl)mercaptopropionate (AC-2, 1.1 eq). The completion of the reaction is determined by HPLC on a reversed phase C18 column as stationary phase and triethylammonium acetate buffer (pH=5.5). The product 3-(S-diphenyl-4-pyridylmethyl)mercaptopropionyl coenzyme A (AC-3) is purified by HPLC, and identified by mass spectral analysis. The product is lyophilized for long term storage.

### iv. Preparation of the 3-mercapto-propionyl Coenzyme A (AC-4)

A solution of 3-(S-diphenyl-4-pyridylmethyl)mercaptopropionyl coenzyme A (AC-3) in acetic acid was added mercuric acetate. The reaction is stirred at room temperature for 15 minutes. The completion of the reaction is determined by HPLC on a reversed phase C18 column as stationary phase and triethylammonium acetate buffer (pH=5.5). The reaction mixture is diluted with HPLC buffer, purified by HPLC, and identified by Mass spectral analysis. The product, 3-mercapto-propionyl coenzyme A (AC-4), is used immediately to prepare the target biosensor.

### v. Preparation of bis-iodoacetyl-hydrazine (AC-5)

To a solution of hydrazine in Dry DMF is added succinimidyl 2-iodoacetate and triethylamine as base. The reaction is monitored by thin layer chromatography on silica gel plates. After complete acylation of hydrazine, DMF is removed under high vacuum. The final (AC-5) is purified by silica gel chromatography, and identified by NMR spectroscopy.

### vi. Preparation of iodoacetyl-AcylCoA, ^{AcI}Acyl-CoA (AC-6)

To a solution of 3-mercapto-propionyl coenzyme A (AC-4) in DMF is added excess if bis-iodoacetylhydrazine (AC-5). The completion of the reaction is monitored by HPLC. The product iodoacetyl-acylCoA (AC-6) is purified by HPLC. The final product is isolated by HPLC, using a C18 reverse phase column as the stationary phase and water (containing triethylammonium acetate pH 5.5-6.0): acetonitrile mixture as the mobile phase.

### 4. Synthesis of the Fluorescent Biosensor for PCAF, _{Cy3B}^{Cy5}FS^{PCAF} (AC-7—coupling PM^{Cy5}•H4^{cy3B} to ^{AcI}Acyl-CoA (AC-6)

FIG. 7B shows the final PCAF AMS. To a solution of PM^{Cy5}•H4^{cy3B} in sodium phosphate buffer (pH 6.0) is added a freshly prepared solution of ^{AcI}Acyl-CoA in sodium phosphate buffer (pH 6.0). Progress of the reaction is monitored by HPLC. The final product

_{Cy3B}^{Cy5}FS^{PCAF} (AC-7) is purified and isolated by HPLC using a C18 reverse phase column as the stationary phase and water (containing triethylammonium acetate pH 5.5-6.0): acetonitrile mixture as the mobile phase. The isolated purified peptide is analyzed by ESI-MS. The proper fractions are lyophilized for use as a biosensor reagent as histone acetyl transferase reagent.

FIG. 8 shows atomic components of a generalized small-molecule component of various embodiments of an AMS. Substitutions at B, X₁, X₂, X₃, X₄, R₁, R′₁, R₂, R′₂, R₃, and R′₃ are possible.

Although the present invention has been described in terms of a particular embodiment, it is not intended that the invention be limited to this embodiment. Modifications within the spirit of the invention will be apparent to those skilled in the art. For example, an artificial, multifunctional substrate can be designed to bind to, and be modified by, any biopolymer-substrate-modifying transferase. Different types of report moieties that produce different types of signals that either strengthen or diminish during the course of a transferase-mediated reaction can be employed in the AMS. While the described embodiments employ two reporter moieties, additional reporter moieties may be included in an AMS.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

### SEQUENCE PROGRAM LISTING APPENDIX

Two identical CDs identified as “Copy 1 of 2” and “Copy 2 of 2,” containing the sequence listing for the present invention, is included as a sequence listing appendix

### 8. Determination of Assay Specificity and Selectivity Using H89 and 1RS727 as Inhibitors of PKA and IRK

To determine the specificity of the assay, H89 (Upstate biotechnology, Waltham, Mass.) with an IC₅₀ of ˜50 nM, and 1RS727 (a peptide inhibitor with the sequence KKKLPATGDYMNMSPVGD) SEQ ID: 1 with a reported Kₘ of ˜24 μM (20), are used as potent competitive inhibitors of PKA and IRK, respectively. The experiments are performed with incubating each enzyme with varying concentrations of the appropriate inhibitor for 30 minutes, and performing the assay as previously described and the inhibition quantitated using the modified Lineweaver-Burke plot of equation 2 for competitive inhibition:

$$\frac{1}{v} = \frac{1}{{V}_{max}} + \frac{{K}_{M}}{{V}_{max} [ S ]} ( 1 + \frac{[ I ]}{{K}_{i}} )$$

where [I] is the concentration of the competitive inhibitor in the experiment; and Kᵢ is the inhibition constant.

### SEQUENCE PROGRAM LISTING APPENDIX

1. A method for producing an artifical, multifunctional substrate for use in a biopolymer-modifying transferase assay, the transferase removing a chemical group from a small-molecule substrate and adding the chemical group to a biopolymer substrate, the method comprising:
selecting a small-molecule-substrate component that binds to a small-molecule-substrate active site of the transferase;
selecting a biopolymer-substrate-mimetic component that binds to a biopolymer-substrate active site of the transferase;
selecting a conformationally flexible linker component that, when attached to the selected small-molecule-substrate component and selected biopolymer-substrate-mimetic component, correctly spaces the selected small-molecule-substrate component from the selected biopolymer-substrate-mimetic component for mutual binding to the small-molecule-substrate active site of the transferase and the biopolymer-substrate active site of the transferase, respectively;
binding the selected small-molecule-substrate component to one end of the linker and binding the selected biopolymer-substrate-mimetic component to a second end of the linker component to produce the artifical, multifunctional substrate.

2. The method of claim 1 further including covalently attaching two reporter moieties to the small-molecule-substrate component/linker-component/biopolymer-substrate-mimetic-component assembly so that the reporter moieties the reporter moieties produce a detectable signal after the artifical, multifunctional substrate is modified by the transferase that is not produced by the reporter moieties prior to modification of the artifical, multifunctional substrate.

3. The method of claim 2 wherein the reporter moieties produce a detectable signal before the artifical, multifunctional substrate is modified by the transferase that is not produced by the reporter moieties after modification of the artifical, multifunctional substrate.

4. The method of claim 1 wherein the small-molecule substrate is a functional-group-donating substrate, the chemical group one of:
a methyl group;
a ubiquinyl group;
a phosphate group;
a glycosyl group;
a sulfate-containing group;
a substituted sulphate group
a substituted phosphate group;
an acetyl group; and
an alkyl group.

5. The method of claim 1 wherein the biopolymer substrate is one of:
a deoxyribonucleic-acid substrate;
a ribonucleic-acid substrate;
a protein substrate;
a polysaccharide substrate;
a glycoprotein substrate; and
a lipid.

6. A method for assaying a transferase-containing solution for transferase activity, the method comprising:
selecting an artifical, multifunctional substrate that includes a small-molecule component linked to a biopolymer-mimetic component by a conformationally flexible linker component, the artifical, multifunctional substrate further including at least one reporter moiety that produces a detectable signal in a first modification state of the artifical, multifunctional substrate, and that does not produce the detectable signal in a second modification state of the artifical, multifunctional substrate;
to a solution containing the transferase, adding the artificial, multifunctional substrate; and
detecting a change, over time, in the detectable signal as the transferase modifies the artifical, multifunctional substrate.

7. The method of claim 6 wherein the at least one reporter moiety produces the detectable signal after the artifical, multifunctional substrate is modified by the transferase.

8. The method of claim 6 wherein the at least one reporter moiety produces a detectable signal before the artifical, multifunctional substrate is modified by the transferase.

9. The method of claim 6 wherein the small-molecule substrate is a functional-group-donating substrate, the chemical group one of:
a methyl group;
a ubiquinyl group;
a phosphate group;
a glycosyl group;
a sulfate-containing group;
a substituted sulphate group;
a substituted phosphate group;
an acetyl group; and
an alkyl group.

10. The method of claim 1 wherein the biopolymer substrate is one of:
a deoxyribonucleic-acid substrate;
a ribonucleic-acid substrate;
a protein substrate;
a polysaccharide substrate;
a glycoprotein substrate; and
a lipid.

11. An artificial, multifunctional substrate that binds to, and that is chemically modified by, a transferase for use in a transferase assay, the transferase having a binding site for a small-molecule, chemical-group-donating substrate and a binding site for a biopolymer substrate to which the transferase adds a chemical group removed by the transferase from the small-molecule, chemical-group-donating substrate, the artificial, multifunctional substrate comprising:
a small-molecule-substrate component;
a biopolymer-mimetic component;
a linker component to which the small-molecule-substrate component and the biopolymer-mimetic component are covalently bound, the conformationally flexible linker having a length and conformational flexibility that allows the small-molecule-substrate component to bind to the small-molecule-substrate binding site of the transferase and the biopolymer-mimetic component to bind to the biopolymer-substrate binding site of the transferase; and
at least two reporter moieties that together produce a detectable signal in a first artificial-multifunctional-substrate-chemical-modification state and that do not produce the detectable signal in a second artificial-multifunctional-substrate-chemical-modification state.

12. The artificial, multifunctional substrate of claim 11 wherein the linker component is equal to or greater than 3 Å in length.

13. The artificial, multifunctional substrate of claim 11 wherein the linker component is equal to or greater than 10 Å in length.

14. The artificial, multifunctional substrate of claim 11 wherein the linker component is equal to or greater than 30 Å in length.

15. The artificial, multifunctional substrate of claim 11 wherein the linker component is equal to or greater than 80 Å in length.

16. The artificial, multifunctional substrate of claim 11 wherein the linker component is comprised of a linear or cyclic chemical chain consisting of a combination of hetero-atoms including C, N, O, P, S, or a combination of linear or cyclic monomers from an organic, synthetic, or biological source, including glycine, β-alanine, ω-amino-α-carboxy-ethylene glycol.

17. The artificial, multifunctional substrate of claim 11 wherein the linker component includes at least one donor/acceptor functional point of attachment, donor/acceptor functional point of attachments including: amine, ester, amide, ether, acetyl, ketal, amidate, carbamate, carbonate, alcohol, Diels-Alder, -ene/-diene, and thiol groups.

18. The artificial, multifunctional substrate of claim 17 wherein a reporter moiety is covalently bound to at least one donor/acceptor functional point of attachment of the linker component.

19. The artificial, multifunctional substrate of claim 11 wherein reporter moieties are fluorophore/quencher moieties with overlapping excitation and emission spectra.

20. The artificial, multifunctional substrate of claim 19 wherein the artificial, multifunctional substrate includes at least one pair of reporter moieties selected from among:
Cy5/Cy3B;
Cy5/Dabcyl;
Cy3B/Dabcyl;
and other dye pairs with overlapping excitation and emission spectra.

21. The artificial, multifunctional substrate of claim 11 wherein the transferase is protein kinase A and the biopolymer-substrate-mimetic component is a minimum consensus peptide sequence Leu-Arg-Arg-Ala-Ser-Leu-Gly (SEQ ID: 2), the Ser residue of which is phosphorylated by protein kinase A.

22. The artificial, multifunctional substrate of claim 11 wherein the transferase is insulin receptor kinase and the biopolymer-substrate-mimetic component is a minimum consensus peptide sequence for insulin receptor kinase, Lys-Lys-Lys-Leu-Pro-Ala-Thr-Gly-Asp-Tyr-Met-Asn-Met-Ser-Pro-Val-Gly-Asp (SEQ ID: 3), the Tyr residue of which is phosphorylated by insulin receptor kinase.

23. The artificial, multifunctional substrate of claim 11 wherein the transferase is one of a serine protein kinase, a threonine protein kinase, and a tyrosine protein kinase, and the biopolymer-substrate-mimetic component is a minimum consensus peptide sequence for the transferase having at least four amino-acid subunits.

24. The artificial, multifunctional substrate of claim 11 wherein the transferase is PCAF histone acetyltransferase and the biopolymer-substrate-mimetic component is a 30 residue N-terminal histone H3 sequence, Ala-Arg-Thr-Lys-Gln-Thr-Ala-Arg-Lys-Ser-Thr-Gly-Gly-Lys-Ala-Pro-Arg-Lys-Gln-Leu-Ala-Thr-Lys-Ala-Ala-Arg-Lys-Ser-Ala-Pro (SEQ ID: 4), the Lys 14 residue of which is acetylated by PCAF histone acetyltransferase.

25. The artificial, multifunctional substrate of claim 11 wherein the transferase is PCAF histone acetyltransferase and the biopolymer-substrate-mimetic component is a 30 residue N-terminal histone H4 sequence for PCAF histone acetyltransferase, Ser-Gly-Arg-Gly-Lys-Gly-Gly-Lys-Gly-Leu-Gly-Lys-Gly-Gly-Ala-Lys-Arg-His-Arg-Lys-Val-Leu-Arg-Asp-Asn-Ile-Gln-Gly-Ile-Thr (SEQ ID: 5), the Lys 8 residue of which is acetylated by PCAF histone acetyltransferase.

26. The artificial, multifunctional substrate of claim 11 wherein the transferase is PRMT-1 and the biopolymer-substrate-mimetic component is a 30 residue N-terminal histone H4 sequence, Ser-Gly-Arg-Gly-Lys-Gly-Gly-Lys-Gly-Leu-Gly-Lys-Gly-Gly-Ala-Lys-Arg-His-Arg-Lys-Val-Leu-Arg-Asp-Asn-Ile-Gln-Gly-Ile-Thr (SEQ ID: 5), the Arg 3 residue of which is methylated by PRMT-1.

27. The artificial, multifunctional substrate of claim 11 wherein the biopolymer-substrate-mimetic component is a library of peptide sequences containing from 4 to 30 contiguous amino acid residues selected from the 30-residue N-terminal histone H4 sequence, Ser-Gly-Arg-Gly-Lys-Gly-Gly-Lys-Gly-Leu-Gly-Lys-Gly-Gly-Ala-Lys-Arg-His-Arg-Lys-Val-Leu-Arg-Asp-Asn-Ile-Gln-Gly-Ile-Thr (SEQ ID: 5).

28. The artificial, multifunctional substrate of claim 27 wherein the amino acid residues within the library of peptide sequences consist of a combination of un-modified as well modified amino acids, including mono-methyl arginine, symmetric and asymmetric di-methyl arginine, mono-methyl lysine, di-methyl lysine, acetyl-lysine, phospho-serine, phospho-threonine, and phosphor-yrosine.

29. The artificial, multifunctional substrate of claim 11 wherein a reporter moiety is covalently bound to a C-terminus of the biopolymer-substrate-mimetic component.

30. The artificial, multifunctional substrate of claim 11 wherein a reporter moiety is covalently bound to an N-terminus of the biopolymer-substrate-mimetic component.

31. The artificial, multifunctional substrate of claim 11 wherein the small-molecule component is a neucleoside or neucleoside analog covalently bound to a linear or cyclic chemical chain consisting of a combination of hetero atoms, the hetero atoms selected from among hetero atoms including C, N, O, P, S, and/or hetero-functional molecules, the hetero-functional molecules selected from among hetero-functional molecules including alkyl, aryl, or a sugar.

32. The artificial, multifunctional substrate of claim 11 wherein the small-molecule component is a nucleoside or nucleoside analog selected from among nucleosides and nucleoside analogs including ATP, GTP, CTP, TTP, ATPγ-S, GTPγ-S, CTPγ-S, and TTPγ-S.

33. The artificial, multifunctional substrate of claim 11 wherein the small-molecule component is a nucleoside analog selected from among tetraphosphate derivatives of ATP, GTP, CTP, TTP, ATPγ-S, GTPγ-S, CTPγ-S, and TTPγ-S.

34. The artificial, multifunctional substrate of claim 11 wherein the small-molecule component is a nucleoside analog including one of: Acetyl-CoA, malonyl-CoA, butyryl-CoA, S-adenosyl-L-methionine, 3′-phosphoadenosine-5′-phosphosulfate (“PAPS”), and nicotinamide adenine di-nucleotide (“NADH”).

35. The artificial, multifunctional substrate of claim 11 wherein the small-molecule component is γ-(2-aminoethyloxy)-ATP.

36. The artificial, multifunctional substrate of claim 11 wherein the small-molecule component is iodoacetyl-acylCoA.

37. The artificial, multifunctional substrate of claim 11 wherein the small-molecule component is S-carboxy-methyladenosyl-homocysteine.

38. The artificial, multifunctional substrate of claim 11 wherein the small-molecule component is a nucleoside or nucleoside analog having the general structure of FIG. 8, wherein base B, is a purine or pyrimidine base selected from among purine and pyrimidine bases including: adenine, cytidine, thymidine, guanine, and derivates of adenine, cytidine, thymidine, and guanine.

39. The artificial, multifunctional substrate of claim 11 wherein the small-molecule component is a nucleoside or nucleoside analog having the general structure of FIG. 8, wherein X₁, X₂, and X₃, and X₄ are any combination of atomic moieties including but not limited to O, S, N, P, or C.

40. The artificial, multifunctional substrate of claim 11 wherein the small-molecule component is a nucleoside or nucleoside analog having the general structure of FIG. 8, wherein the hetero-functional moieties R₁, R₁′, R₂, R₂′, and R₃, R₃′ are any combination of functional moieties selected from among functional moieties including: a lone pair of electrons, phosphorous, sulfur, nitrogen, phosphate, sulfate, nitrate, sulfhydryl, amine, alkyl, aryl, hydrogen, and an organic, synthetic or biological polymer.

41. The artificial, multifunctional substrate of claim 11 wherein the reporter moieties are chromophores and the detectable signal produced by the reporter moieties is a fluorescent-resonance-energy-transfer signal.

42. The artificial, multifunctional substrate of claim 11 wherein the reporter moieties include NMR-detectable atoms the detectable signal produced by the reporter moieties is splitting of NMR peaks.

43. The artificial, multifunctional substrate of claim 11 wherein the reporter moieties combine, when in close proximity, to form a stable association with at least one bond detectable by spectroscopy.
